(19) United States
(12) Patent Application Publication
(10) Pub. No.: US 2024/0382109 A1
(43) Pub. Date: Nov. 21, 2024

(54) SYSTEMS AND METHODS FOR THE AUTOMATED MONITORING OF ANIMAL PHYSIOLOGICAL CONDITIONS AND FOR THE PREDICTION OF ANIMAL PHENOTYPES AND HEALTH OUTCOMES

(71) Applicant: Pig Improvement Company UK Limited, Basingstoke, Hampshire (GB)

(72) Inventors: Eric Psota, Lincoln, NE (US); William Herring, Gallatin, TN (US); Robert Fitzgerald, Lafayette, TN (US)

(73) Assignee: Pig Improvement Company UK Limited, Basingstoke, Hampshire (GB)

(21) Appl. No.: 18/691,444

(22) PCT Filed: Sep. 14, 2022
(86) PCT No.: PCT/GB2022/052322

§ 371 Date: Mar. 12, 2024

Related U.S. Application Data
(60) Provisional application No. 63/244,622, filed on Sep. 15, 2021.
(60) Provisional application No. 63/279,384, filed on Nov. 15, 2021.

(51) Int. Cl. A61B 5/11; A61B 5/00; G16H 50/30

(52) U.S. Cl. CPC A61B 5/112; A61B 5/0077; A61B 5/1128; A61B 5/7275; G16H 50/30; A61B 2503/40

(57) ABSTRACT

The present invention relates to the automated monitoring of animals, in particular livestock animals such as swine, for the identification or determination of particular physical characteristics or conditions that may be used to predict one or more phenotypes or health outcomes for the each of the animals. Systems and methods are provided for the non-subjective and automatic identification or prediction of one or more phenotypes, such as weight or gait, based on computer-vision system analysis of video or image data capture of an animal retaining space in a commercial farming operation. The predicted or identified phenotypes are used to predict one or more health outcomes or scores for an animal.

### FIELD OF THE INVENTION

The present invention relates to the automated monitoring of animals, in particular livestock animals such as swine, for the identification or determination of particular physical characteristics or conditions that may be used to predict one or more phenotypes or health outcomes for the each of the animals.

### BACKGROUND

Animal productivity and health metrics, such as those determined based on observed phenotypes, may be subjective or difficult to quantify by a human observer. Moreover, these types of subjective visual assessments may be time consuming and difficult to accurately correlate or associate with an individual animal by the human observer. For example, some metrics, such as sow productive lifetime or sow longevity for porcine animals, are complex traits that may be influenced or determined by many genetic and environmental factors and which may be difficult to effectively and repeatably quantify using human observers. Identifying and quantifying certain phenotypic characteristics, such as feet and leg soundness, lameness, or leg problems, is important in the field of animal husbandry as issues such as these that may be visually identified by an external examination of an animal represent a significant reason for animals being selected for removal from commercial breeding herds.

Existing work in making visual phenotypic observations relies on identifying specific physical characteristics or relationships between characteristics, and then subjectively determining whether or not an animal matches a desirable or undesirable phenotypic characteristic. Desirable characteristics, as shown in FIGS. 24-25, 27, and 30, provide exemplary representations of observable phenotypes indicative of a positive health condition or outcome related to gait, leg structure, or foot size in gilts or sows for swine animals. FIGS. 26, 28-29, and 31-32 provide exemplary representations of observable phenotypes indicative of a negative or undesirable health condition or outcome related to, respectively, buck kneed, post legged, sickle hocked, uneven length, or small size in gilts or sows for swine animals.

For visual phenotypic measurements to be accurate, repeatable, and useful, they must be able to measure the front/rear leg structure, such as by precisely detecting key anatomical points (e.g., feet, knee, hock, joints, head, shoulder, etc.). However, existing manual methods for making these measurements and observations are imprecise and subjective, and existing studies have not implemented any technologically implemented method capable of discerning the structural features of the leg joints. Some academic studies have attempted to address these deficiencies and have detected the carpal/tarsal joints and hooves using, for example, an object detection algorithm, but in doing so used multiple, expensive time-of-flight cameras to set up the experimental environment. This type of set up is too complex and expensive for commercial applications. Therefore, the existing systems and methods cannot directly identify the necessary phenotypic characteristics, such as identifying a predicted weight or a gait structure in an animal, such as a gilt or sow.

Manual, large-scale phenotyping of animal behavior traits in a manual manner by human observers is time consuming and subjective. However, existing methods for the automated tracking of animals has only seen limited testing in controlled environments and may require the use of expensive or complicated equipment to implement. Additionally, recording from animals in large groups in highly variable farm settings presents challenges.

In some animal husbandry applications, such as on commercial farms, in breeding operations, genetic nucleus farms, and in multiplier farming operations, animal behavior and phenotypic traits may be implicitly used in an informal way every day by farmers and staff to assess the health and welfare of the animals in their care. For example, systematic and quantitative recordings of farm animal behavior may be made by researchers, veterinarians and farm assurance inspectors, who then may manually implement, by visual observation of the recordings or other data, numerical scoring systems to record aspects of injury or lameness. Some phenotypic and behavioral traits may be sufficiently heritable such that genetic selection to modify them may be possible. Therefore, it may be desirable to identify those animals with desirable phenotypic or behavioral traits to be selected or removed from a breeding program, or to identify an animal or animals for a health treatment-type intervention.

The use of cameras to automate the recording of behavior has already been applied to species that are easy to manage in highly-controlled settings, for example movement tracking of color-labelled laboratory rodents in small groups indoors under constant artificial light in standardized caging. Commercial farm conditions offer several challenges including group sizes and stocking density, unmarked individuals, variable lighting and background, and the possibility that the animal becomes soiled with dirt or feces. One of the current key knowledge gaps is how to track individual animals in a pen and record their behavior while continuously recognizing the individual, especially when dealing with unmarked animals without wearable sensors.

In addition to animal behavior, information streams which may be utilized in a commercial farming operation may include sensors which provide information about the farm environment or building control systems such as meteorological information, temperature, ventilation, the flow of water or feed, and the rate of production of eggs or milk. With the development of the Internet of Things (“IoT”), it may be desirable to connect disparate data streams and to combine those data streams with non-subjective assessments of phenotypic traits or physical/anatomical conditions for animals to provide for the optimum outcome for the animals and for a commercial farming operation.

What is needed is an automated computer-vision system capable of identifying key anatomical points (e.g., feet, knee, hock, joints, head, shoulder, etc.). What is needed is a commercially-implementable system capable of extracting and filtering potential gait features based on the spatial relationship of these key points. Furthermore, what is needed is a mathematical model to relate between gait features and the feet/leg soundness.

Additionally, what is needed is an automated computer-vision system capable of identifying individual animals from an image and predicting a phenotype for the animal. What is needed is a commercially-implementable system capable of identifying individual animals and predicting a phenotype, such as longevity based on a predicted weight, based on an image provided by a low-cost image sensor.

### SUMMARY OF THE INVENTION

Provided herein are systems and methods for automatically monitoring one or more animals to derive a phenotype for each of the monitored animals. Animals, such as livestock (e.g., cows, goats, sheep, pigs, horses, llamas, alpacas), may be housed in animal retaining spaces such as pens or stalls that may be disposed within covered structures such as barns. The systems and methods may comprise capturing images or video of animals, such as side-views or from top-down views, while the animals are disposed in the animal retaining spaces or walkways within a barn or other structure. The images may then be stored in a networked video storage system that is in electronic communication with the image sensor, such as a camera, webcam, or other suitable image sensor, located at or near the animal retaining spaces.

Image processing of the images captured by the image sensor and stored at the networked video recorder may be performed by one or more machine learning algorithms, such as a fully convolutional neural network. Anatomical features or segments may be identified for individual animals located with an image frame, and an image processor, such as a suitable configured graphics processing unit implementation of a machine-vision system, may be used to predict or determine one or more phenotypic characteristics associated with an individual animal.

What is provided is a system and method to precisely measure front/rear leg angle. A side-view camera system collects images used in generated 2-D pose estimation models. The system and method locate key body identifying key anatomical points (e.g., feet, knee, hock, joints, head, shoulder, etc.). These points are used to derive a phenotypic characteristic, such as gait pattern and a gait score, that may be used in predicting a health outcome or in determining a health or other animal husbandry action to take with respect to an individual action. What is provided is a system and method which implements machine learning to predict foot and leg score and other animal longevity characteristics from information collected and annotated by an automated computer machine-vision system. The system and method provides for an accurate, repeatable, and non-subjective assessment of one or more phenotypic characteristics of an animal (e.g., gait score, gait pattern, animal longevity, stride length, foot score, leg score) by determining topographical points or a set of anatomical landmarks of the animal from an image or video, and provides an assessment of the phenotypic characteristics using a fully convolutional neural network to predict a health outcome for the animal.

Additionally, without wishing to limit the present invention to any theory or mechanism, it is believed that the methods and systems herein are advantageous because existing systems and methods are not capable or suitable for commercial implementation and use. The systems and methods provided herein implement lower-cost solutions suitable for use in a commercial implementation. The systems and methods provided herein can predict or identify phenotypic characteristics and predict or determine health outcomes for individual animals using images or video captured by “security-camera” or “webcam” type commercially-available image sensors and processed by local or remote (e.g., “cloud-based”) image processing servers implementing fully convolutional neural networks.

In various embodiments, what is provided is a method for deriving a gait pattern in an animal, the method comprising: capturing a set of image frames of the animal, wherein the animal is in motion; determining a location of the animal for each image frame in the set of image frames; identifying a set of anatomical landmarks in the set of image frames; identifying a set of footfall events in the set of image frames; approximating a stride length for the animal based on the location of the animal in each image frame of the set of image frames, the set of anatomical landmarks, and the set of footfall events; and deriving the gait pattern based in part on the stride length, the location of the animal in each image frame of the set of image frames, the set of anatomical landmarks, and the set of footfall events.

In various embodiments, the animal is a swine.

In various embodiments, the set of image frames comprise high-resolution image frames. The high-resolution image frames comprise a resolution of at least 720 p.

In various embodiments, the motion is from a left side to a right side or from the right side to the left side in an image frame form the set of image frames, and wherein the motion is in a direction perpendicular to an image sensor.

In various embodiments, the set of image frames are captured by an image sensor. The image sensor is a digital camera capable of capturing color images. The image sensor is a digital camera capable of capturing black and white images.

In various embodiments, the set of image frames comprise a video.

In various embodiments, the method comprises determining the presence or absence of the animal in an image frame from the set of image frames.

In various embodiments, the method comprises updating a current location of the animal to the location of the animal in an image frame from the set of image frames.

In various embodiments, the method comprises determining a beginning and an end of a crossing event. The crossing event comprises a continuous set of detections of the animal in a subset of the set of image frames. The beginning of the crossing event is determined based in part on identifying that the animal occupies 20% of a left or right portion of an image frame. The end of the crossing event is determined based on identifying that the animal occupies 20% of the opposite of the left or right portion of the image frame from the beginning of the crossing event.

In various embodiments, the set of anatomical landmarks comprise a snout, a shoulder, a tail, and a set of leg joints.

In various embodiments, the method comprises interpolating an additional set of anatomical landmarks using linear interpolation where at least one of the set of anatomical landmarks could not be identified.

In various embodiments, each footfall event in the set of footfall events comprises a subset of image frames wherein a foot of the animal contacts a ground surface.

In various embodiments, approximating the stride length further comprises calculating the distance between two of the set of footfall events.

In various embodiments, the stride length is normalized by a body length of the animal.

In various embodiments, the method comprises computing a delay between a footfall event associated with a front leg of the animal and a footfall event associated with a rear leg of the animal. The method further comprises deriving a stride symmetry based in part on the delay. Deriving the gait pattern is based in part on the stride symmetry.

In various embodiments, deriving the gait pattern is based in part on a head position of the animal in a walking motion.

In various embodiments, deriving the gait pattern is based in part on a set of leg angles.

In various embodiments, the method comprises predicting a phenotype associated with the animal based on the derived gait pattern. The phenotype comprises a future health event associated with at least one leg of the animal. The method further comprises selecting the animal for a future breeding event based on the phenotype. The method further comprises identifying the animal as unsuitable for breeding based on the phenotype. The method further comprises subjecting the animal to a medical treatment based on the phenotype. The health treatment is a surgery. The health treatment is removal from a general animal population. The health treatment is an antibiotic treatment regimen. The health treatment is culling the animal.

In various embodiments, the method comprises reading identification tag associated with the animal. The capturing the set of image frames is triggered by the reading of the identification tag.

In various embodiments, the identifying the set of anatomical landmarks in the set of image frames further comprises: processing each image frame in the set of image frames using a fully convolutional neural network; identifying a nose, a mid-section, a tail, and a set of joints of interest using the fully convolutional neural network; producing a set of Gaussian kernels centered at each of the nose, the mid-section, the tail, and the set of joints of interest by the fully convolutional neural network; and extracting the set of anatomical landmarks as feature point locations from the set of Gaussian kernels produced by the fully convolutional neural network using peak detection with non-max suppression.

In various embodiments, identifying the set of anatomical landmarks in the set of image frames further comprises interpolating an additional set of anatomical landmarks, the interpolating comprising: identifying a frame from the set of image frames where at least one anatomical landmark from the set of anatomical landmarks is not detected; and interpolating a position of the at least one anatomical landmark by linear interpretation between a last known location and a next known location of the at least one anatomical landmark in the set of image frames to generate a continuous set of data points for the at least one anatomical landmark for each image frame in the set of image frames.

In various embodiments, the trained classification network is trained based in part on the stride length, the location of the animal in each frame in the set of image frames, the set of anatomical landmarks, and the set of footfall events. The trained classification network is further trained based on a delay between footfall events in the set of footfall events, a set of leg angles, a body length of the animal, a head posture of the animal, and a speed of the animal in motion. The gait score represents a time the animal is expected to be in use before culling.

In various embodiments, the method comprises: transmitting the set of image frames to a network video recorder; and storing the set of images on the network video recorder.

In various embodiments, the method comprises identifying the set of anatomical landmarks in the set of image frames by an image processing server.

In various embodiments, the method comprises identifying the set of footfall events in the set of image frames by an image processing server.

In various embodiments, the method comprises approximating the stride length for the animal based on the location of the animal in each image frame of the set of image frames, the set of anatomical landmarks, and the set of footfall events by an image processing server.

In various embodiments, the method comprises deriving the gait pattern based in part on the stride length, the location of the animal in each image frame of the set of image frames, the set of anatomical landmarks, and the set of footfall events by an image processing server.

In various embodiments, what is provided is a method of predicting at least one health outcome for an animal, the method comprising: capturing a set of high-resolution image frames of the animal, wherein the animal is in motion during the capture of the set of high-resolution image frames, and wherein the set of high-resolution image frames are captured at a rate of at least sixty times per second; determining a presence of the animal in each frame from the set of high-resolution image frames; determining a location of the animal within each frame from the set of high-resolution image frames; setting a tracked animal location as the location of the animal in a first frame in the set of high-resolution image frames where the presence of the animal is determined; updating the tracked animal location for each frame in each frame from the set of high-resolution image frames to generate a sequence of tracked animal locations; identifying a beginning and an end of an event based on the sequence of tracked animal locations, the beginning of the event comprising a first frame from the set of high-resolution image frames wherein the tracked animal location for the first frame is disposed in a left or right portion of the first frame, and the end of the event comprising a second frame from the set of high-resolution image frames wherein the tracked animal location for the second frame is disposed in an opposite portion of the second frame relative to the first frame, and wherein each frame in the set of high-resolution image frames from the first frame to the second frame comprises a set of event frames; identifying a first set of anatomical landmarks of the animal for each frame in the set of event frames; interpolating a second set of anatomical landmarks for the animal for each frame in the set of event frames, wherein the second set of anatomical landmarks comprise anatomical landmarks not in the first set of anatomical landmarks; identifying a set of footfall events from the set of event frames, a footfall event comprising a subset of frames wherein a foot of the animal contacts a ground surface; approximating a stride length for the animal based on a distance between footfall events in the set of footfall events and normalizing the stride length for the animal based on a determined body length of the animal; determining a delay between a set of front leg footfalls and a set of rear leg footfalls in the set of footfall events; deriving the gait pattern based in part on the stride length, the set of footfall events, the first set of anatomical landmarks, and the second set of anatomical landmarks, the gait pattern comprising the stride length, a symmetry of stride, a speed, a head position, and a set of leg angles; and determining a future health event for the animal based on the gait pattern, wherein the future health event is associated with an identified deficiency, abnormality, or inconsistency identified in the gait pattern.

In various embodiments, what is provided is a method of estimating a phenotypic trait of an animal, the method comprising: capturing a top-down image of the animal; bounding and isolating a central portion of the image, the central portion comprising a least distorted portion of the image; identifying a center of a torso of the animal; cropping the central portion of the image at a set distance from the center of the torso of the animal to form a cropped image; segmenting the animal into at least head, shoulder, and torso segments based on the cropped image; concatenating the at least head, shoulder, and torso segments onto the cropped image of the animal to form a concatenated image; and predicting a weight of the animal based on the concatenated image.

In various embodiments, the animal is a swine.

In various embodiments, the image comprises a greyscale image.

In various embodiments, the image comprises a set of images. The set of images comprises a video.

In various embodiments, image is captured by an image sensor. The image sensor is a digital camera. The image sensor is disposed at a fixed height with a set of known calibration parameters. The known calibration parameters comprise a focal length and a field of view. The known calibration parameters comprise one or more of a saturation, a brightness, a hue, a white balance, a color balance, and an ISO level.

In various embodiments, central portion comprising the least distorted portion of the image further comprises a portion of the image that is at an angle substantially perpendicular to a surface on which the animal is disposed.

In various embodiments, identifying the center of the torso of the animal further comprises tracking an orientation and location of the animal using a fully convolutional neural network.

In various embodiments, the method comprises extracting an individual identification for the animal. The extracting the individual identification for the animal further comprises reading a set of identification information from a tag disposed on the animal. The tag is an RFID or a visual tag. The extracting of the set of identification information is synchronized with the capturing of the top-down image.

In various embodiments, the cropping the central portion of the image at the set distance from the center of the torso of each of the animal further comprises: marking the center of the torso of the animal with a ring pattern; and cropping the central portion of the image at the set distance to form the cropped image. The set distance is 640×640 pixels.

In various embodiments, the segmenting the animal into the at least head, torso, and shoulder segments further comprises segmenting the animal into at least left and right head segments, left and right shoulder segments, left and right ham segments, and left and right torso segments based on the center of the torso for the animal.

In various embodiments, segmenting the animal into the at least head, torso, and shoulder segments further comprises segmenting by a fully convolutional neural network. The fully convolutional neural network is trained on an annotated image data set.

In various embodiments, segmenting is based on a ring pattern overlaid on the animal based on the center of the torso of the animal. No output may be produced where the ring pattern is not identified.

In various embodiments, the concatenating comprises stacking the at least head, shoulder, and torso segments on the cropped image in a depth-wise manner to form the concatenated image. The concatenated image comprises an input into a deep regression network adapted to predict the weight of the animal based on the concatenated image. The deep regression network comprises 9 input channels. The 9 input channels comprise the cropped image as a channel and 8 body part segments each as separate channels. The method further comprises augmenting the training of the deep regression network by randomly adjusting the position, rotation, and shearing of a set of annotated training images.

In various embodiments, the method comprises predicting a phenotype associated with the animal based on the weight of the animal. The phenotype comprises a future health event associated with the animal. The method further comprises selecting the animal for a future breeding event based on the phenotype. The method further comprises identifying the animal as unsuitable for breeding based on the phenotype. The method further comprises subjecting the animal to a medical treatment based on the phenotype. The health treatment is a surgery. The health treatment is removal from a general animal population. The health treatment is an antibiotic treatment regimen. The health treatment is culling the animal.

In various embodiments, the weight of the animal represents a time the animal is expected to be in use before culling.

In various embodiments, what is provided is method of estimating a weight of an animal based on a set of image data, the method comprising: capturing a top-down, greyscale image of at least one animal by an electronic image sensor, the electronic image sensor disposed at a fixed location, a fixed height, and with a set of known calibration parameters; bounding and isolating a central portion of the image, the central portion comprising a least distorted portion of the image that is at an angle substantially perpendicular to a surface on which the at least one animal is disposed; identifying a center of a torso of each of the at least one animal using a fully convolutional neural network; cropping the central portion of the image at a set distance from the center of the torso of each of the at least one animal; segmenting each of the at least one animal into at least left and right head segments, left and right shoulder segments, and left and right torso segments based on the center of the torso for each of the at least one animal; concatenating the at least left and right head segments, left and right shoulder segments, and left and right torso segments onto the top-down image of each of the at least one animal to form a set of concatenated images; and predicting a weight for each of the at least one animal based on the set of concatenated images.

In various embodiments, what is provided is system for determining a phenotypic trait of an animal based on a set of captured image data, the system comprising: a camera mounted above an animal retaining space and disposed at a fixed height above a central location in the animal retaining space, the camera adapted to capture and transmit an image of an animal; a horizontally-mounted camera disposed at a height aligned with a shoulder height of the animal and at an angle perpendicular to a viewing window, the horizontally-mounted camera adapted to capture and transmit a set of image frames of the animal, wherein the animal is in motion; a tag reader disposed proximate to the animal retaining space, the tag reader adapted to read a tag associated with the animal and to transmit a set of identification information read from the tag; a network video recorder comprising a storage media, the network video recorder in electronic communication with the horizontally-mounted camera and adapted to: receive the image transmitted from the camera; receive the set of image frames transmitted from the horizontally-mounted camera; and store the set of image frames and the image on the storage media; an image processing server comprising a processor and a memory, the image processing server in electronic communication with the network video recorder, and the memory comprising a first set of computer-executable instructions that when executed by the processor are adapted to cause the image processing server to automatically: request and receive the set of image frames from the network video recorder; determine a location of the animal for each image frame in the set of image frames; identify a set of anatomical landmarks in the set of image frames; identify a set of footfall events in the set of image frames; approximate a stride length for the animal based on the location of the animal in each image frame of the set of image frames, the set of anatomical landmarks, and the set of footfall events; derive the gait pattern based in part on the stride length, the location of the animal in each image frame of the set of image frames, the set of anatomical landmarks, and the set of footfall events; and store the gait pattern, the stride length, the location of the animal in each image frame of the set of image frames, the set of anatomical landmarks, and the set of footfall events in a first database, wherein each of the gait pattern, the stride length, the location of the animal in each image frame of the set of image frames, the set of anatomical landmarks, and the set of footfall events are associated with the set of identification information read from the tag; the image processing server comprising a second set of computer-executable instructions that when executed by the processor are adapted to cause the image processing server to automatically: request and retrieve the image from the network video recorder; bound and isolate a central portion of the image, the central portion comprising a least distorted portion of the image; identify a center of a torso of the animal; crop the central portion of the image at a set distance from the center of the torso of the animal; segment the animal into at least head, shoulder, and torso segments; concatenate the at least head, shoulder, and torso segments onto the top-down image of the animal to form a concatenated image; predict a weight of the animal based on the concatenated image; and store the predicted weight of the animal in a second database; and wherein a predicted phenotype for the animal is derived from the predicted weight and the gait pattern.

In various embodiments, what is provided is a system for deriving a gait pattern in an animal, the system comprising: a horizontally-mounted camera disposed at a height aligned with a centerline of the animal and at an angle perpendicular to an animal viewing window, the horizontally-mounted camera adapted to capture and transmit a set of image frames of the animal, wherein the animal is in motion; a tag reader disposed proximate to a walking path, the tag reader adapted to read a tag associated with the animal and to transmit a set of identification information read from the tag; a network video recorder comprising a storage media, the network video recorder in electronic communication with the horizontally-mounted camera and adapted to: receive the set of image frames transmitted from the horizontally-mounted camera; and store the set of image frames on the storage media; an image processing server comprising a processor and a memory, the image processing server in electronic communication with the network video recorder, and the memory comprising a set of computer-executable instructions that when executed by the processor are adapted to cause the processor to automatically: request and receive the set of image frames from the network video recorder; determine a location of the animal for each image frame in the set of image frames; identify a set of anatomical landmarks in the set of image frames; identify a set of footfall events in the set of image frames; approximate a stride length for the animal based on the location of the animal in each image frame of the set of image frames, the set of anatomical landmarks, and the set of footfall events; derive the gait pattern based in part on the stride length, the location of the animal in each image frame of the set of image frames, the set of anatomical landmarks, and the set of footfall events; and store the gait pattern, the stride length, the location of the animal in each image frame of the set of image frames, the set of anatomical landmarks, and the set of footfall events in a database, wherein each of the gait pattern, the stride length, the location of the animal in each image frame of the set of image frames, the set of anatomical landmarks, and the set of footfall events are associated with the set of identification information read from the tag.

In various embodiments, what is provided is system for estimating a weight of an animal, the system comprising: a camera mounted above an animal retaining space and disposed at a fixed height above a central location in the animal retaining space, the camera adapted to capture and transmit an image of an animal of one or more animals; a network video recorder comprising a storage media, the network video recorder in electronic communication with the camera and adapted to: receive the image transmitted from the camera; and store the image on the storage media; an image processing server comprising a processor and a memory, the image processing server in electronic communication with the network video recorder, and the memory comprising a set of computer-executable instructions that when executed by the processor are adapted to cause the processor to automatically: request and retrieve the image from the network video recorder; bound and isolate a central portion of the image, the central portion comprising a least distorted portion of the image; identify a center of a torso of the animal; crop the central portion of the image at a set distance from the center of the torso of the animal; segment the animal into at least head, shoulder, and torso segments; concatenate the at least head, shoulder, and torso segments onto the top-down image of the animal to form a concatenated image; predict a weight of the animal based on the concatenated image; and store the predicted weight of the animal in a database.

In various embodiments, what is provided is an animal health monitoring system, the system comprising: a plurality of image sensors, wherein a first image sensor from the plurality of image sensors is disposed above an animal retaining space, and wherein a second image sensor from the plurality of image sensors is disposed facing a side of the animal retaining space, the side of the animal retaining space comprising a view of the animal retaining space, the plurality of image sensors adapted to capture and transmit a set of images of the animal retaining space; a network video recorder comprising a storage media, the network video recorder in electronic communication with the plurality of image sensors and adapted to: receive the set of images from the plurality of image sensors; and store the set of images on the storage media; a phenotype prediction server comprising a processor and a memory, the phenotype prediction server in electronic communication with the network video recorder, and the memory comprising a set of computer-executable instructions that when executed by the processor are adapted to cause the processor to automatically: request and retrieve the set of images from the network video recorder; process the set of images using a fully convolutional neural network to identify a center point of the animal; identify a set of physical characteristics and anatomical landmarks of the animal based in part on the identified center point of the animal; predict a set of phenotypes associated with the animal based on the set of physical characteristics and anatomical landmarks; and present the set of phenotypes to a user in graphical user interface.

In various embodiments, what is provided is an automated smart barn, the smart barn comprising: an animal retaining space disposed in the smart barn for holding at least one animal, the animal retaining space comprising a supporting surface and a set of retaining walls; a walking path adjoining the animal retaining space, the walking path comprising a viewing widow providing a view of the walking path; a tag reader disposed proximate to the walking path, the tag reader adapted to read a tag associated with the animal and to transmit a set of identification information read from the tag, the set of identification information associated with the animal; a plurality of image sensors, wherein a first image sensor from the plurality of image sensors is disposed above the animal retaining space, and wherein a second image sensor from the plurality of image sensors is disposed facing the viewing window, the plurality of image sensors adapted to capture and transmit a set of images of the animal in the animal retaining space or walking path; a network video recorder comprising a storage media, the network video recorder in electronic communication with the plurality of image sensors and adapted to: receive the set of images from the plurality of image sensors; and store the set of images on the storage media; a phenotype prediction server comprising a processor and a memory, the phenotype prediction server in electronic communication with the network video recorder, and the memory comprising a set of computer-executable instructions that when executed by the processor are adapted to cause the processor to automatically: request and retrieve the set of images from the network video recorder; process the set of images using a fully convolutional neural network to identify a center point of the animal; identify a set of physical characteristics and anatomical landmarks of the animal based in part on the identified center point of the animal; predict a set of phenotypes associated with the animal based on the set of physical characteristics and anatomical landmarks; and present the set of phenotypes and the set of identification associated with the animal to a user in graphical user interface.

The various embodiments of systems and methods provided herein provide for improvements to the functioning of a computer system by enabling faster and more accurate machine vision-based identification and prediction of phenotypic traits and prediction and determination of health outcomes by a fully convolutional neural network that is less expensive and less computationally intensive than can be provided by any existing system or method, and which improves on, and provides significant capabilities which are not possible through, any manual or human-provided system or method.

### BRIEF DESCRIPTION OF THE DRAWINGS

In order to facilitate a full understanding of the present invention, reference is now made to the accompanying drawings, in which like elements are referenced with like numerals. These drawings should not be construed as limiting the present invention but are intended to be exemplary and for reference.

FIG. 1 provides a diagram representing a system for determining a gait pattern for an animal based on side-view image capture according to one embodiment.

FIG. 2 provides a flow chart of steps for a method for determining a gait pattern for an animal based on side-view image capture according to one embodiment.

FIGS. 3 and 4 provide photographic side-view images of an animal with a set of anatomical landmarks overlaid on the animal for determining a gait pattern for the animal based on the side-view image capture according to one embodiment.

FIGS. 5 and 6 provide graphical representations of stride length distributions of a set of gait scores according to one embodiment.

FIG. 7 provides a diagram representing a system for determining a predicted weight for an animal based on top-down image capture according to one embodiment.

FIG. 8 provides a flow chart of steps for a method for determining a predicted weight for an animal based on top-down image capture according to one embodiment.

FIG. 9 provides a photographic top-down image of animals in animal retaining spaces, such as pens and walkways, before and after segmentation processing by a fully convolutional neural network for determining a predicted weight for the individual animals according to one embodiment.

FIG. 10 provides a photographic top-down image of animals in an animal retaining space, after identification and segmentation processing by a fully convolutional neural network for determining a predicted weight for the individual animals wherein predicted and measured weights are overlaid on the animals, according to one embodiment.

FIGS. 11-22 provide graphical representations of predicted weights and measured weights for a set of animals over defined time periods according to one embodiment.

FIG. 23 provides a block diagram of a system for identifying or predicting a phenotype for an animal based on information collected by one or more sensors and processed by an image processing module according to one embodiment.

FIGS. 24-32 provide line-drawing illustrations of desirable and undesirable phenotypes associated with the legs and feet of swine.

### DETAILED DESCRIPTION

The systems and methods herein will now be described in more detail with reference to exemplary embodiments as shown in the accompanying drawings. While the present invention is described herein with reference to the exemplary embodiments, it should be understood that the systems and methods herein are not limited to such exemplary embodiments. Those possessing ordinary skill in the art and having access to the teachings herein will recognize additional implementations, modifications, and embodiments, as well as other applications for use of the invention, which are fully contemplated herein as within the scope of the systems and methods as disclosed and claimed herein, and with respect to which the systems and methods herein could be of significant utility.

In one embodiment, the systems and methods herein provide automated pig structure and gait detection by the automated, objective, structured phenotyping and the translation of the phenotypic to keep/cull decisions for sows, boar, and gilts based on the predicted phenotypes (e.g., gait pattern or score).

In one embodiment, to predict the phenotype, (e.g., provide a gait estimation, gait pattern, and/or gait score) a system and method comprises capturing a high-resolution, side-view video or set of images at 60 Hz or greater of an animal (e.g., pig) in motion, for example while walking through an alleyway or walkway. For each frame of the images or video, the presence or absence of a pig of interest is determined for each frame. A location is also determined for the pig of interest in the frame. If the current location is near the location of the last detection, the current location of a tracked pig is updated. Using the sequence of tracked locations, it is identified when a pig crosses the field of view, and the beginning and end of the crossing event are marked as comprising a continuous set of detections from either left-to-right or right-to-left from the set of images or video. The beginning of the event is defined as when the pig of interest enters either the 20% most left or right portion of the view. The end of the event is defined as when the pig exits the opposite 20% side of view relative to the beginning of the event. At the conclusion of a tracking event, the current location is reset and a new pig can be tracked. For each frame of a tracking event, the locations of the snout, shoulder, tail, and all easily identifiable leg joints are identified. When anatomical landmarks are not identified, their locations are “filled-in” or interpolated using linear interpolation between existing anatomical landmark detections. Foot falls are identified as events where one of the four feet of the pig of interest makes contact with the ground for at least a predetermined number of consecutive frames. The distance between foot falls is calculated to approximate stride length and the stride length is normalized by body length of the pig of interest. A delay between foot falls of the front and rear legs is computed based on a number of image frames or a duration of video between the determined foot fall events. The determined delay is indicative of weight shifting and favoritism towards healthy or strong legs and of overall symmetry of the gait. The stride length, symmetry of stride, speed, head position while walking, and a set of determined leg angles are used to predict future health events related to the pigs' legs as assessed by a gait pattern and gait score derived from the images by a fully convolutional neural network.

The system, in one embodiment, comprises a side-view security camera positioned perpendicular to a viewing window for a walkway or alleyway which provides for the capture of a set of images or video of an animal of interest in motion (e.g., walking across the viewing window from left-to-right or right-to left). The camera is positioned at a height (e.g., 2-3 feet off of the ground) such that both left and right-side legs of the animal are visible. The camera is connected to a Network Video Recorder (“NVR”) co-located at the same site or location as the camera. The NVR receives the images captured and transmitted by the camera for storage and later processing. A server, such as an image processing server comprising a graphics processing unit (“GPU”), is connected to the NVR via a secure file transfer protocol (“SFTP”). The server and NVR may also be co-located with the camera. The server may be a GPU-powered embedded computer such as an NVIDIA JETSON. The image processing server is configured to request, receive, and process video captured by the camera and recorded by the NVR to extract a gait pattern and a gait score for individual pigs. An API such as those provided in MATLAB, TENSORFLOW, or PYTORCH, or similar API or software package or environment capable of implementing deep learning, computer vision, image processing, and parallel computing, may be used to implement a trained fully convolutional neural network for image processing. In one embodiment, car tag IDs are read using RFID and are transmitted to the image processing server using a BLUETOOTH connection. In another embodiment, visual tags are read by an image sensor and information is extracted using a machine vision-based system. The gait or leg score generated by the image processing server are stored locally or in a remote database and are provided to a user via a local or web-based graphical user interface.

In determining a gait pattern, gait score, or leg score, the video or set of images used therein is trimmed based on the identification tag being read and based on the location of the animal (pig) of interest in a frame. Once the tag (e.g., RFID tag or a visual tag) is read from the car tag on the pig and is transmitted to the image processing server, a process is started using the body part detection network (fully convolutional neural network) to look for a pig of interest to enter the frame. Once the pig enters, its body center is tracked across the frame until it exits the field of view. This encapsulates a walking event video with associated with a read ID from a tag associated with the animal. To detect joints or anatomical landmarks in a walking event for a pig of interest, each frame of the trimmed video or set of images is processed with a deep joint detection network to detect the nose, mid-section, tail, and leg joints of interest. In some embodiments, a YOLOv3 object detection model is applied to isolate animals, such as gilts, from the background image.

The network used to detect joint positions is a deep, fully-convolutional network that produces Gaussian kernels centered at each joint position. The fully-convolutional neural network comprises three deconvolution layers which may determine a pose estimation by stacking the three deconvolution layers. The variance of the kernels represents the uncertainly of human annotation, so that precise body parts have small kernels and ill-defined areas like the center of the mid-section have wide kernels. Feature point locations are extracted from the network outputs using peak detection with non-max suppression. The stacking of the three deconvolution layers by the fully-convolutional neural network is used to extract the location of body landmarks. For example, using the three deconvolution layers by the fully-convolutional neural network to extract the location of body landmarks, 19 body landmarks were extracted with a mean average precision (“mAP”) of 99.1%.

To interpolate missing anatomical landmarks or joints, frames without a detection are filled via interpolation to form a complete and continuous data point. This interpolation method marks the first and last appearance of a joint in a sequence of frames and interpolates all missing locations between these frames. Specifically, linear interpolation is used to fill the gaps so that, for example, if frame 2 and 5 had detections but 3 and 4 did not, the interpolated position of the joint for frame 3 would be two thirds of the position of frame 2 and one third of the position in frame 5. The interpolated position for frame 4 would be one third of the position of frame 2 and two thirds of the position of frame 5. This method results in smooth movements throughout the frame sequence. To provide a gait pattern or score, which may be associated with or used to derive a foot or a leg score, the positions of the joints or anatomical landmarks, included interpolated anatomical landmarks, are processed to extract meaningful parameters like stride length, delay between from and back foot falls, leg angles, body length, head posture, and speed. These data points are then used to train a classification network to score the pig. The target for scoring is a prediction or measure of the duration of time the pig is expected to be in use before identified leg issues cause the pig to be culled or removed from use. The scoring may also be used to identify or flag the animal for one or more health treatments based on a type of defect or abnormality that is phenotypically identified for the animal.

In one embodiment, static features such as stride length and leg angle, and dynamic features such as lagging indicator and skeleton energy image are extracted and evaluated based on the anatomical landmarks extracted from the image by the fully convolutional neural network. A combination of features, such as leg angle and lagging indicator, may provide better performance relative to a single feature such that animals comprising the best and worst gaits are linearly separable. Additionally, an extracted or determined stride length may be used as a key feature to compare against manual or visually determined scores. A kernel density plot provides that stronger legs with higher leg scores generally produce longer strides.

In one embodiment, the systems and methods herein provide automated prediction of individual weights for swine using consumer-grade security or webcam type image sensor footage of animal retaining spaces such as pens based on the application of a fully convolutional neural network to identify individual animals and concatenate segmented body portions onto depth-corrected cropped portions of an original image.

In one embodiment, to provide a predicted phenotype, for example an estimated weight, a system and method comprises capturing video or a set of images (e.g., image frames) from a top-down mounted camera with a fixed height and with known camera calibration parameters. The known height and image projection process ensures that pig's weight is reflected in the image in a consistent manner. The center portion of the image with a determined lowest level or amount of lens distortion and comprising the most top-down view is identified. For pigs that overlap with the center portion or which are fully located within the center portion, the center location of the pigs' torsos in the video are identified using a fully convolutional neural network. For each detected pig the center location is marked with a ring pattern, and then a 640×640 image is cropped around that pig to form a cropped image. The cropped image is fed into another, separate, fully convolutional neural network to segment 8 body parts, the 8 body parts comprising the left/right ham, left/right torso, left/right shoulder, and left/right head. The segmented image produced by the segmentation network is concatenated with the original grayscale image and fed into a deep regression network to predict a weight for the animal.

The system, in one embodiment, comprises an overhead security camera connected via power-over-ethernet (“PoE”) for power and data to a Network Video Recorder (“NVR”) co-located at the same site or location as the camera. The NVR receives the images captured and transmitted by the camera for storage and later processing. A server, such as an image processing server comprising a graphics processing unit (“GPU”), is connected to the NVR via a secure file transfer protocol (“SFTP”). The image processing server is configured to request, receive, and process video captured and recorded by the camera to extract weight information for individual pigs. An API such as those provided in MATLAB, TENSORFLOW, or PYTORCH, or similar API or software package or environment capable of implementing deep learning, computer vision, image processing, and parallel computing, may be used to implement a trained fully convolutional neural network for image processing.

Short term tracking of individual animals within the weight estimation area, which resides in the center of the frame, is achieved by extracting each pig's location and orientation from an image frame by a fully convolutional neural network. The fully convolutional neural network may comprise a stacking of three or more deconvolution layers. Individual identification for an animal is extracted in one of two ways, however, other ways of identifying and extracting identification information for individual animals may also be implemented. In a machine vision-based embodiment, an car tag identifying an animal is detected and read using a classifier neural network. In a radio tag-based embodiment, an RFID reader is disposed in or near the animal retaining area, such as proximate to a feeder or drinker, and the animals individual identification information is read and transmitted to the NVR or image processing server in-sync with the video feed to link detections to individual identification information. For body part segmentation, after location and identification for an animal of interest are established, the body parts of an animal of interest (e.g., a pig of interest) are segmented using a fully-convolutional neural network to identify the locations of left and right side rear, mid, shoulder, and head body segments. The fully convolutional neural network is trained using over 3000 examples of segmented pigs obtained via human annotation. The pig of interest is marked in the input image by placing a visual ring pattern on the mid-section of the pig. This provides for the network to recognize and differentiate the individual pig of interest from all other pigs in the image. When no ring pattern is present, the network is trained to produce an output that contains only unused background. To estimate the weight of the animal (pig), the original image, which may be a greyscale image, is stacked or concatenated with the segmentation output depth-wise to form the input to a deep regression network that estimates the weight. Therefore, the input to the weight estimation network contains 9 channels comprising the grayscale image as one channel and 8 channels body segment channels with 1's indicating the presence of each associated body part (0's at all other locations in the image). Training augmentation is used when training the network to randomly adjust position, rotation, and shearing to improve the accuracy of the weight estimation. No scale adjustments are applied so that the scale stays consistent and can be used by the network for prediction.

Weight estimates are stored locally or in a remote database, such as one managed by a cloud services provider. The weight estimates and other phenotypic information or predictions are provided to a user through a locally accessible or web-based graphical user interface (“GUI”).

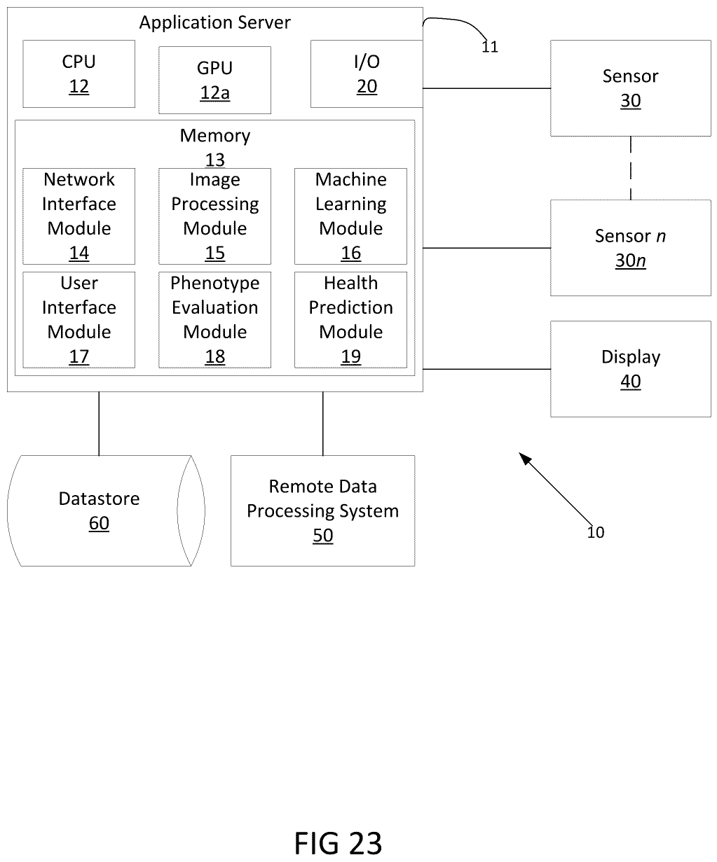

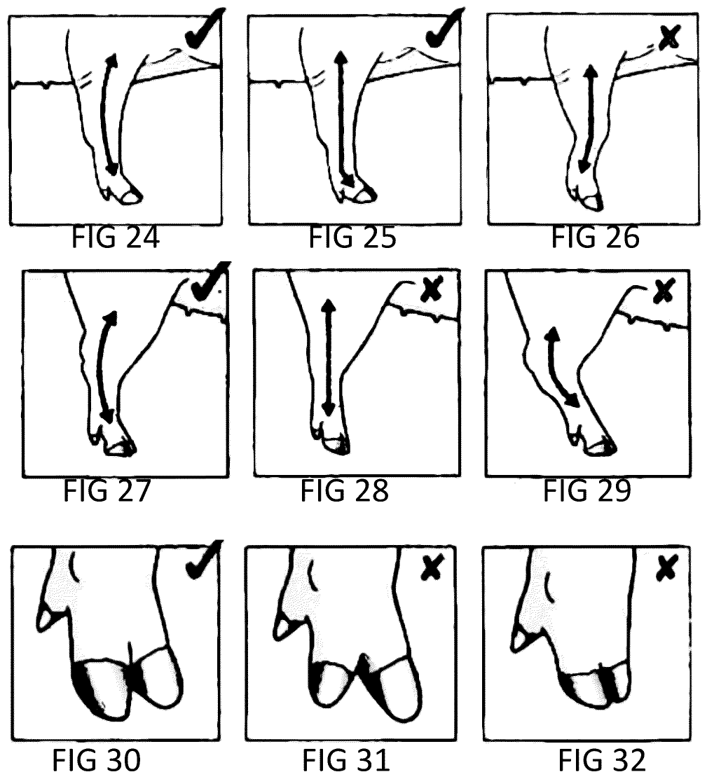

Now, with respect to FIG. 23, a block diagram of a system 10 for identifying or predicting a phenotype for an animal based on information collected by one or more sensors 30 and processed by an image processing module 15 according to one embodiment is provided. The system 10 comprises an application server 11, a set of sensors 30 to 30n, a display 40, a remote data processing system 50, and a datastore 60. The application server 11 is a specially-configured computer system comprising a CPU 12, a GPU 12a, an input/output (“I/O”) interface 20, and a memory 13. A set of specially configured modules are stored in the memory 13, which may be a non-transitory computer readable media. The modules comprise a network interface module 14, an image processing module 15, a machine learning module 16, a user interface module 17, a phenotype evaluation module 18, and a health prediction module 19. The identified modules may be separate modules configured to, when executed, cause the CPU 12 or GPU 12a to perform specific functions, and may be separate modules or may have their functionality shared or combined in varying embodiments.

The sensors 30 through 30n comprise a set of sensors connected to the application server 11 through electronic communications means, such as by Ethernet or BLUETOOTH connections. The sensors 30 through 30n may comprise sensors such as image sensors (e.g., electronic video cameras or CCD cameras), RFID readers, pressure sensors, weight sensors, or proximity sensors. The I/O module 20 receives communications or signals from the sensors 30 through 30n where they may be directed to the appropriate module within the application server 11.

The datastore 60 is a remote database or data storage location, such as an NVR, where data may be stored. In one embodiment, one or more of the sensors 30 through 30n are in direct communication with the datastore 60. The datastore 60 may be a remote database or data storage service such as a cloud storage provider that may be used to store and manage large volumes of data, such as images, video, phenotype predictions, or other information collected or processed by the system 10.

The remote data processing system 50 may share or comprise some or all of the functions of the application server 11, thereby offloading some or all of the functions to a more suitable location where necessary. For example, some functions may be too processor or computationally intensive or expensive to be co-located with an animal retaining space, such as at a commercial farm. In these circumstances, it may be desirable to move some or all of the more computationally expensive or intensive activities off-site to be performed by the remote data processing system 50, which may be owned and operated by the user of the application server 11, or may be owned and operated by a third-party services provider.

With respect to the modules of the application server 11, the network interface module 14 provides for the handling of communication between the sensors 30 through 30n, the datastore 60, the remote data processing system 50, and the application server 11, such as through Ethernet, WAN, BLUETOOTH, or other wired or wireless radio telecommunications protocols or methods. The network interface module 14 may handle the scheduling and routing of network communications within the application server 11. The user interface module 17 provides for the generation of a GUI which may display predicted phenotypic information or health predictions or outcomes. Other information processed or stored in the server 11, or remotely accessible via the datastore 60 or remote data processing system 50, may also be presented to a user via a GUI generated by the user interface module 17. The user interface module may be used to generate locally viewable or web-based GUIs which may be used to view information on the application server 11 or to configure the parameters of the any system module.

The image processing module 15, which may be a module configured to provide for computer-based and GPU driven machine vision, comprises a deep learning or fully convolutional neural network that is trained and configured as described above. The machine learning module 16 provides for the input and configuration of training data that is used to train and establish the deep learning or fully convolutional neural network implemented by the image processing module 15. The image processing module 15 is configured to receive as input one or more images, image frames, or video data, such as data stored in the datastore 60, to process the images such that the phenotype evaluation module 18 and health prediction module 19 may make determinations as to actual or predicted phenotypes or health outcomes derived from the image data processed by the image processing module 15. For example, side-view or top-view image data captured and stored as described above may be fed into the trained fully convolutional neural network as input, and a set of anatomical landmarks or body segments may be identified from the input image data by the fully convolutional neural network. The phenotype evaluation module 18 may then identify or predict one or more phenotypes, such as a prediction weight or a gait pattern, based on output of the image processing module 15. The output of the phenotype evaluation module 18 may then be used by the health prediction module 19 to predict one or more health outcomes for an animal, such as longevity, and may also be used to recommend or provide a notification related to a health outcome altering action, such as medical attention or culling. The health outcome may also be the suggested inclusion in, or removal from, a breeding program.

The display 40 is in electronic communication with the application server 11 and may provide for the viewing of a GUI displaying predicted phenotypic information or health predictions or outcomes. Other information processed or stored in the server 11, or remotely accessible via the datastore 60 or remote data processing system 50, may also be presented to a user via a GUI in the display 40. In some embodiments, the display 40 is associated with a separate computer or computing device, such as a smartphone, tablet, laptop, or desktop computer which is used by a user to remotely view and access the application server 11.

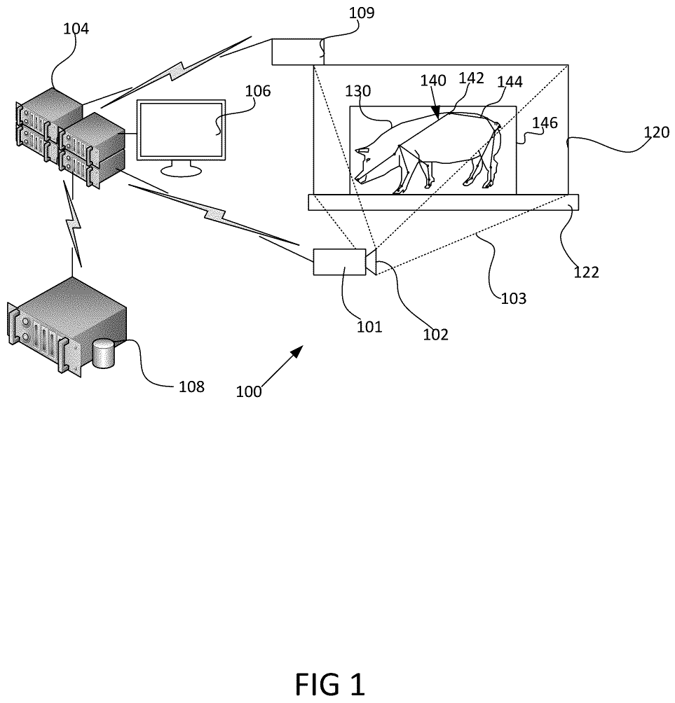

With reference now to FIG. 1, a diagram representing a system 100 for determining a gait pattern for an animal 130 based on side-view image capture according to one embodiment is provided. The system 100 comprises an image capture device 101, such as an electronic or CCD camera, having a lens 102, a tag reader 109, an application server 104, a display 106 and a remote server 108. The image sensor 101 and the tag reader 109 are in electronic communication, such as via Ethernet or a wireless radio communication link such as BLUETOOTH, with the application server 104, which is in electronic communication, such as by local area network or wide area network (e.g., Internet), with the remote server 108.

The application server 104 may be one or more special purpose computing devices, such as an NVR and an image processing server comprising a GPU, and in some embodiments the functionality of the application server 104 may be distributed among a plurality of local machines and/or to the remote server 108, which may be one or more computing devices, or may be a cloud computing or storage solution or service.

The image sensor 101 is positioned such that the a field of view 103 of the lens 102 is pointed or directed towards a viewing area or window 120 of a walkway or alleyway 122 through or over which the animal 130 may traverse, such as by a walking or running motion.

In operation, as the animal 130 traverses the walkway 122 past the viewing window 120 images or video of the animal are captured by the image sensor 101 and transmitted to the application server 104, and at approximately the same time the tag reader 109, which may be an RFID, NFC, or other wireless tag reader, or a visual type tag reader capable of reading a visual tag comprising images, characters, numerals, or other fiducials, reads a set of identification stored in a tag associated with or disposed on the animal 130.

At the application server 104, which may be co-located in the same facility as the image sensor 101 and animal 130, or may be located in a remote facility, the images are processed by a fully convolutional neural network to identify a set of anatomical landmarks 140 for the animal 130 based on a location of the animal within an image frame 146. The set of anatomical landmarks 140 comprises a set of joints or vertices 142 and a set of connecting edges 144 used to define the animal 130 within the frame 146. A central location of the animal 130 is used to locate a central portion of the animal's torso within the frame 146. The changes in the set of anatomical landmarks 140 over a plurality of image frames, comprising a tracking or detection event having a beginning and an end, are used to determine, by a fully convolutional neural network, a gait pattern or structure for the animal 130.

The determined gait pattern or structure may further be used to determine or predict one or more other phenotypic traits or characteristics for the animal such as stride length, delay between from and back foot falls, leg angles, body length, head posture, and speed. The determined gait pattern or the phenotypic characteristics or traits may further be used to predict or determine a health outcome or prediction for the animal such as longevity, foot and leg score, lameness, or disease.

Using tag-based identifiers or other identification means, such as a machine-vision system, to individually identify each animal 130 that traverses the walkway 122 provides for the system 100 to individually provide gait patterns, phenotype predictions or determinations, or health outcomes or predictions for each individual animal.

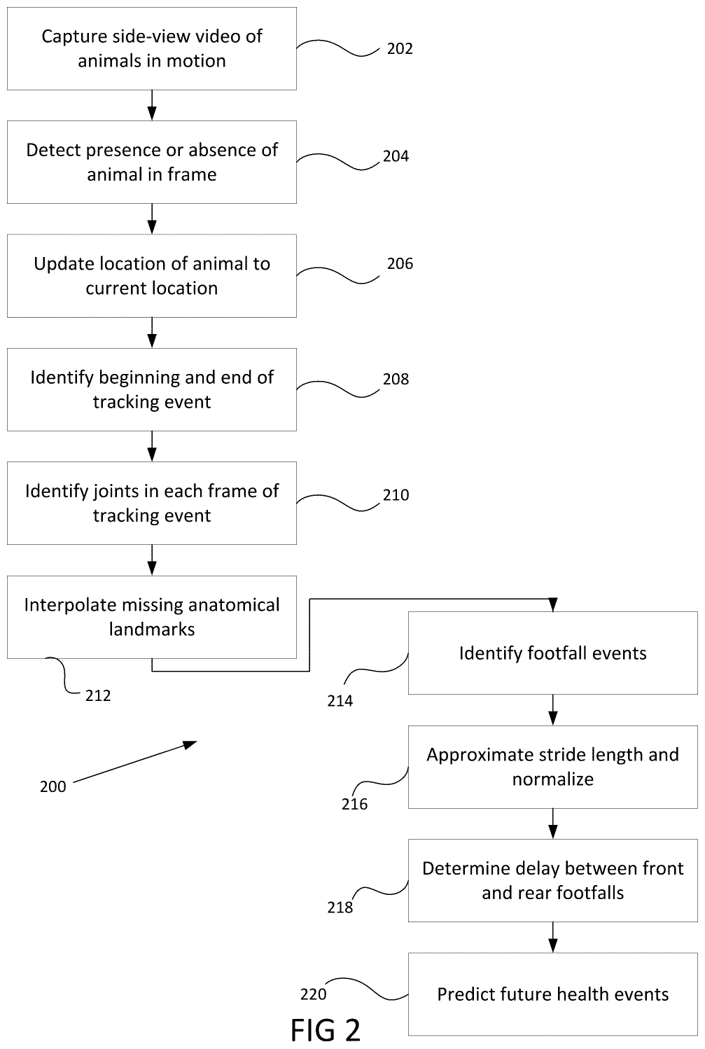

With reference now to FIG. 2, a flow chart 200 of steps for a method for determining a gait pattern for an animal based on side-view image capture according to one embodiment is provided. In step 202, images or video of an animal in motion are captured using a side-view video capture system (e.g., a webcam or other electronic camera capable of capturing images or video at a rate of at least 60 frames per second) positioned such that a view of a walkway traversed by the animal is provided. In step 204, the presence or absence of an animal in each frame of the captured video is determined. In step 206, the current location of the animal is updated to be the location of the animal in each individual frame based on a determined central location of a torso of the animal by a fully convolutional neural network. In step 208, the beginning and end of a tracking event are identified. The beginning of a tracking event is where the animal is determined to occupy at least 20% of a right or left side of a frame, and the end of a tracking event is where the animal is determined to occupy 20% of an opposite of the side of the frame that initiated the beginning of the tracking event. In step 210, individual joints, or anatomical landmarks such as face, snout, shoulder, leg joints, torso, and tail, are identified using a fully convolutional neural network. In step 212, the position of any anatomical landmark which was not identified in an individual frame is interpolated based on the position of the landmark in one or more proceeding or following image frames. In step 214, a set of footfall events for the animal are identified based on identifying a number of frames in which a foot of the animal contacts a surface of the walkway during the walking motion. In step 216, a stride length is approximated based on the footfall events and the identified anatomical landmarks. The stride length may be normalized based on a determined body length for the animal. In step 218, a delay is determined between a front footfall event and a rear footfall event for the motion. The delay may be used to identify abnormalities or defects in the stride such as favoring a side or leg, unequal stride length, or other defect or injury. In step 220, one or more future health outcomes or events are determined or predicted based on one or more of a derived gait pattern, a gait score, a foot and leg score, stride length, delay between from and back foot falls, leg angles, body length, head posture, speed, longevity, lameness, and useful life.

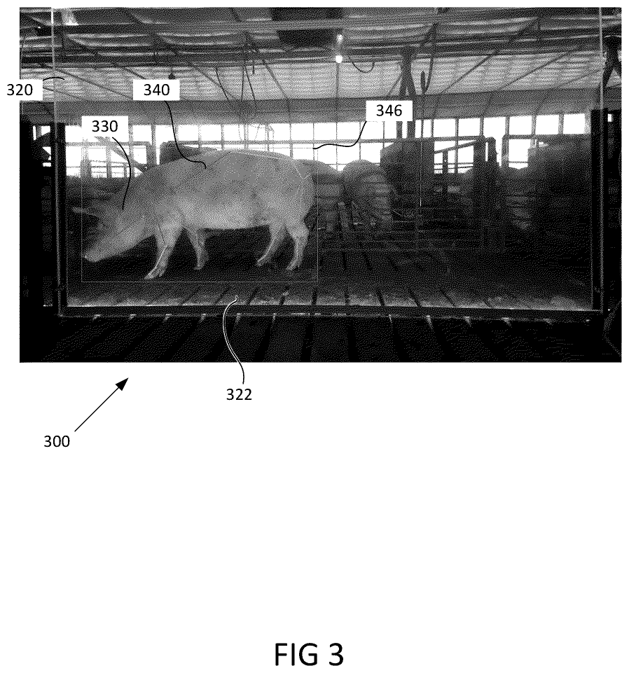

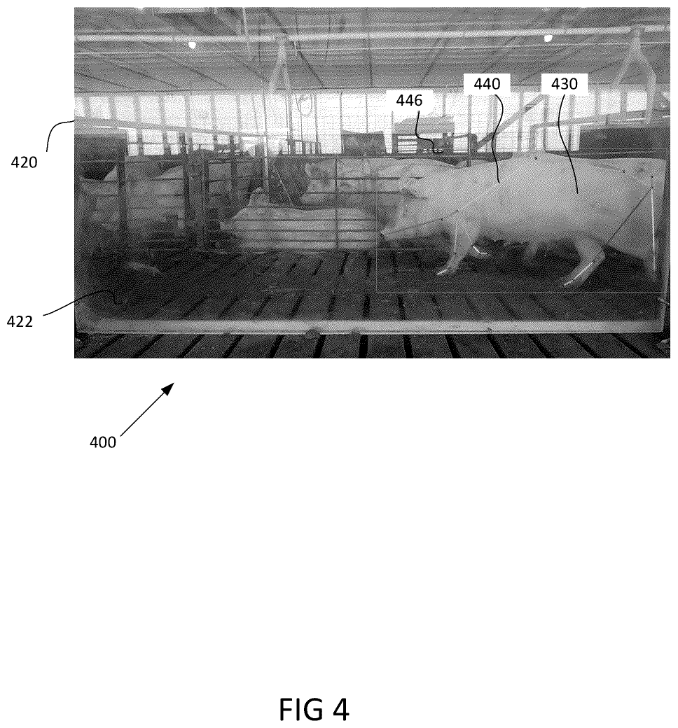

With reference now to FIGS. 3 and 4, photographic side-view images (300, 400) of an animal (330, 430) with a set of anatomical landmarks (340, 440) overlaid on the animal for determining a gait pattern for the animal based on the side-view image capture according to one embodiment are provided. In the images (300, 400), the animals (330, 430) can be seen in motion traversing a walkway (322, 422) past a viewing window (320, 420). A set of anatomical landmarks (340, 440) are shown overlaid on the animals (330, 430) at the location of the animal within the frame (346, 446). In both FIGS. 3 and 4, the anatomical landmarks were generated using the fully convolutional neural network and other systems and methods described herein, and may be used to predict or determine other phenotypic characteristics or traits, or to identify or predict one or more health outcomes or conditions.

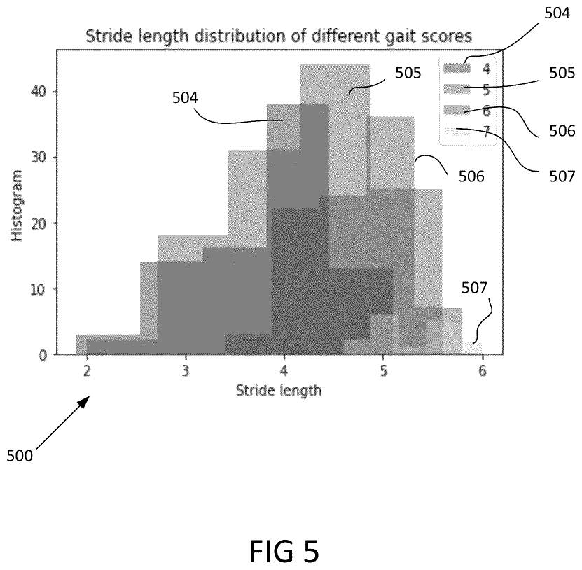

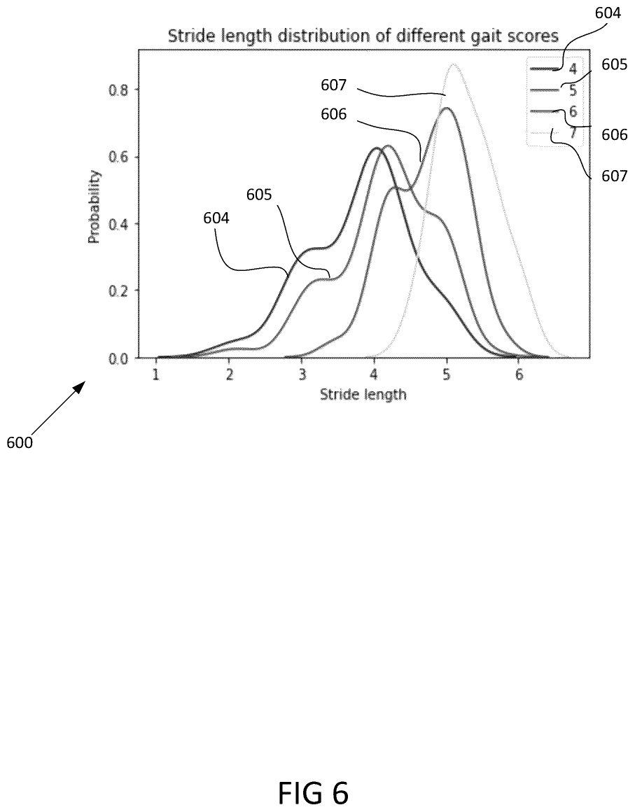

With reference now to FIGS. 5 and 6, graphical representations of stride length distributions of a set of gait scores according to one embodiment are provided. In FIG. 5, a set of gait scores 4 (504), 5 (505), 6 (506), and 7 (507), are shown in a graph 500 of gait score results derived, such as by the system 100 of FIG. 1, having a vertical axis of a number of occurrences and a horizontal axis of a stride length, generally illustrating that higher scores are less frequent and are more frequently associated with a longer stride length. In FIG. 6, a set of gait scores 4 (604), 5 (605), 6 (606), and 7 (607), are shown in a graph 600 of gait score results derived, such as by the system 100 of FIG. 1, having a vertical axis of probability of occurrence and a horizontal axis of a stride length, generally illustrating that higher scores are more commonly associated with a longer stride length. Features, scores or outcomes other than a foot or leg score may be derived from a gait pattern, stride length, or delay.

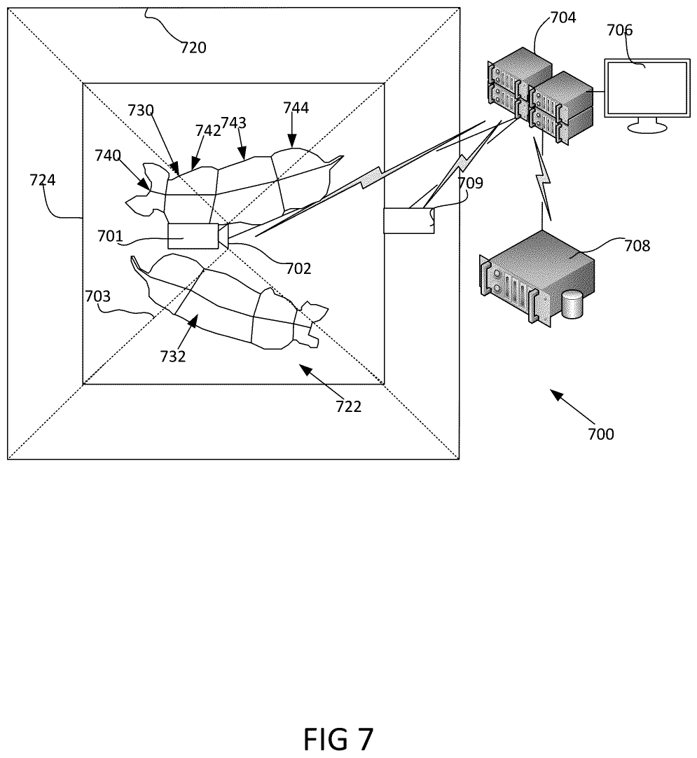

With reference now to FIG. 7, a diagram representing a system 700 for determining a predicted weight for an animal based on top-down image capture according to one embodiment is provided. The system 700 comprises an image capture device 701, such as an electronic or CCD camera, having a lens 702, a tag reader 709, an application server 704, a display 706 and a remote server 708. The image sensor 701 and the tag reader 709 are in electronic communication, such as via Ethernet or a wireless radio communication link such as BLUETOOTH, with the application server 704, which is in electronic communication, such as by local area network or wide area network (e.g., Internet), with the remote server 708.

The application server 704 may be one or more special purpose computing devices, such as an NVR and an image processing server comprising a GPU, and in some embodiments the functionality of the application server 704 may be distributed among a plurality of local machines and/or to the remote server 708, which may be one or more computing devices, or may be a cloud computing or storage solution or service.

The image sensor 701 is positioned such that the field of view 703 of the lens 702 is pointed or directed towards an animal retaining space 720 (e.g., a pen) where a first animal 730 and a second animal 730 are disposed. The retaining space 720 may be defined by a plurality of enclosing walls, which may have one or more openings, gates, or doors, and by a supporting floor or surface, and which may have an open or unenclosed top.

In operation, when the animals 730 and 732 are positioned in a generally central location or central portion 722 of the animal retaining space 720, images or video of the animals 730 and 732 are captured by the image sensor 701 and transmitted to the application server 704, and at approximately the same time the tag reader 709, which may be an RFID, NFC, or other wireless tag reader, or which may be a visual tag reader, may read a set of identification stored in a tag associated with or disposed on the animal 730.

At the application server 704, which may be co-located in the same facility as the image sensor 701 and animals 730 and 732, or may be located in a remote facility, the images are processed by a fully convolutional neural network to identify a central bounding location 724 of each image frame. Within the central bounding location 724, a center of a torso for each of the animal 730 and 732 are identified. A ring pattern is superimposed on each of the animals based on the identified center, and sub images or cropped images are generated based on the identified centers and ring patterns by a fully convolutional neural network. After the cropped images are generated, body segments are generated for each animal. For example, left and right head segments 740, left and right shoulder segments 742, left and right torso segments 743, and left and right butt or ham segments 744 are generated by a fully convolutional neural network for the animal 730. The body segments and the cropped images are concatenated together to form a concatenated image, and the concatenated image is used as an input for another fully convolutional neural network to predict a weight for the animal.

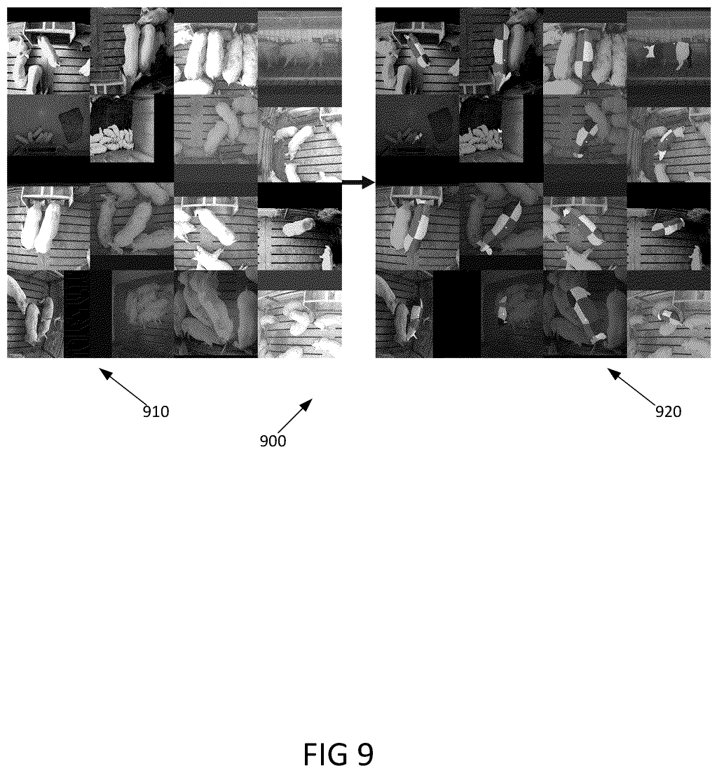

For example, as shown in the images 900 of FIG. 9, a set of input images in color and greyscale 910 and corresponding images segmented by the fully convolutional network 920 are provided. Specifically the images 900 of FIG. 9, provide photographic top-down and side-view images 910 of animals in animal retaining spaces, such as pens and walkways, before and after segmentation processing 920 by a fully convolutional neural network for determining a predicted weight for the individual animals. In some embodiments, the images captured and used as input for a fully convolutional neural network may be color images, black and white images, depth images, 2-D images, 3-D images, or thermal images captured by a correspondingly suited image sensor device.

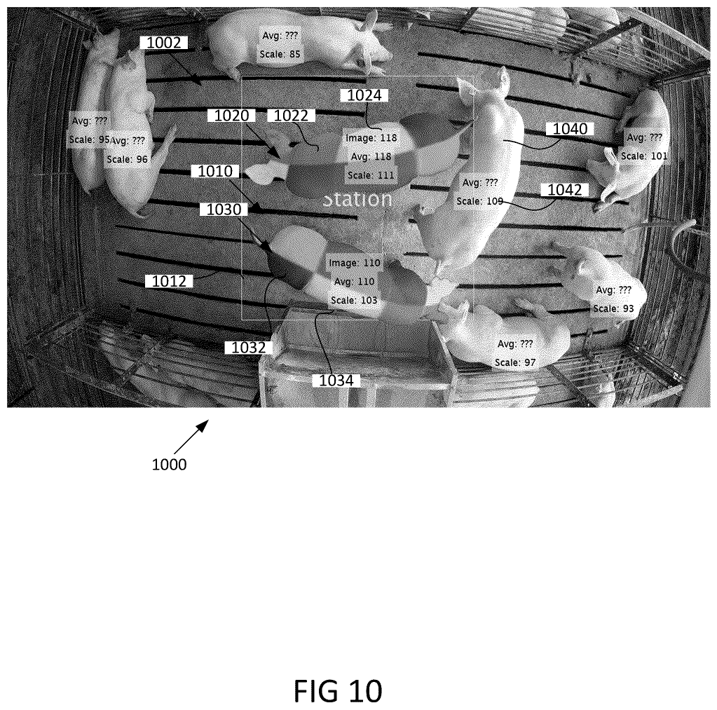

With reference now to FIG. 10, a photographic top-down image 1000 of animals 1020, 1030, and 1040 in an animal retaining space 1002, after identification and segmentation processing by a fully convolutional neural network for determining a predicted weight for the individual animals and wherein predicted and measured weights are overlaid on the animals is provided. As shown in the image 1000, a predicted weight was only determined for the animals 1020 and 1030 which were fully in the central area 1010 of the image defined by the boundary 1012 which comprises an area of least image distortion and which is substantially perpendicular to the supporting surface or ground of the animal retaining space 1002. Body segmentation 1022 and 1032, of the respective animals 1020 and 1030, is shown overlaid or concatenated on the greyscale image of the animals. Additionally, a predicted weight derived from the image by the fully convolutional neural network is overlaid on each of the animals 1020 and 1030 with an actual weight as determined by a scale or other physical measurement device. A scale weight 1042 is shown for the animal 1040, but as the animal was outside the central portion 1010 of the image frame, no predicted weight was derived for the animal 10402, or for any other animal in the image frame and outside of the central portion 1010.

With reference back to FIG. 7, the determined weight prediction may further be used to determine or predict one or more other phenotypic traits or characteristics for the animal such as longevity or useful life. The predicted weight or the phenotypic characteristics or traits may further be used to predict or determine a health outcome or prediction for the animal. Using tag-based identifiers or other identification means, such as a machine-vision system, to individually identify each animal 730 or 732 within the animal retaining space 720 provides for the system 700 to individually provide gait patterns, phenotype predictions or determinations, or health outcomes or predictions for each individual animal.

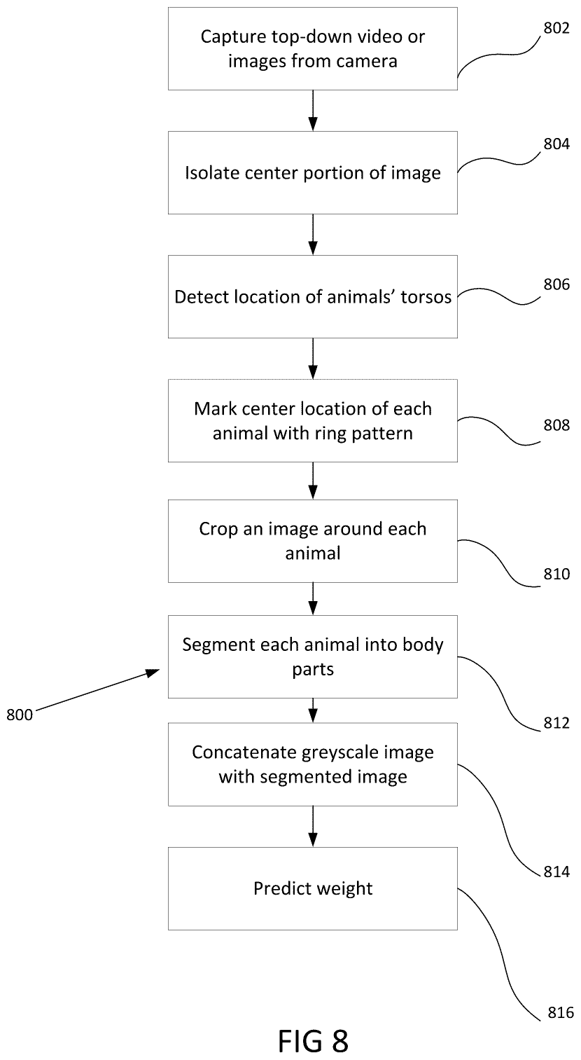

With reference now to FIG. 8, a flow chart of steps for a method for determining a predicted weight for an animal based on top-down image capture according to one embodiment is provided. In step 802, images or video of animals in a retaining space are captured by an image sensor, such as a camera, oriented top-down relative to the retaining space. The camera is positioned at a known or fixed high relative to the retaining space and is configured using a known set of camera configuration parameters such as ISO level, focal length, lens type, white balance, color balance, hue, saturation, bit rate, frame rate, and shutter speed. In step 804, the central portion of each image frame is isolated. The central portion of each image frame is a portion of the image frame comprising the lowest level of lens distortion and which comprises a portion of the floor, ground, or supporting surface of the animal retaining space which is the most substantially perpendicular to a lens of the image sensor. In step 806, the central location of each of the animals' torsos is determined for each animal within the central portion of the image frame by the fully convolutional neural network. In step 808, each central location of the animals' torsos is identified or marked with a ring pattern. In step 810, the image is cropped around each marked ring pattern at a set distance, such as by 640×640 pixels, to generate a set of one or more cropped images, with one cropped image corresponding to each identified animal within central portion of the image frame, but no output is generated where no marked ring pattern is identified. In step 812, the fully convolutional neural network segments the body of each animal in each cropped image into a set of body part segments such as a left and right head body part section, a left and right should body part section, a left and right torso body part section, and a left and right butt, ham, or tail body part section. In step 814, the segmented body part sections are concatenated with the cropped, greyscale images to form a set of concatenated images. In step 816, the set of concatenated images are used as input into a fully convolutional neural network for predicting a weight for each animal. Additional phenotypic characteristics or health outcomes may also be derived from the concatenated images or predicted weights, such as a useful life for the animal, a heath intervention action, additional feeding, inclusion or removal from a breeding program, or a culling action for the animal.

In one embodiment, what may be provided is an automated system and method for the monitoring of animal physiological conditions and for the prediction of animal phenotypes and health outcomes. The system may comprise the system 10 provided in FIG. 23, and may comprise both the gait detection system 100 provided in FIG. 1, and the weight measurement system 700 provided in FIG. 7. A smart-barn, or animal housing structure comprising a plurality of animal retaining spaces such as pens, may be set up and configured to capture visually observable phenotypic information for the prediction or estimation of other phenotypic characteristics or traits to be used in the identification or prediction of health outcomes for the animals in the barn. Various cameras and other sensors may be configured above and near the pens within the barn, and the sensors may be connected via various network connection protocols to local or cloud-based image storage servers, such as NVRs, and image processing servers, which may be one or more application servers. The data captured in the pens of the smart-barn by the sensors is recorded, transmitted, and stored for further processing. Individual identification of animals in the animal retaining spaces may be achieved through tag-based identification means, or may be achieved through a machine vision-based system. Machine learning algorithms, such as fully convolutional neural networks or deep learning networks, may be trained on human annotated input images for the automated processing of the input data. Based on one or more automated processing steps by the fully convolutional neural networks, a set of predicted or identified phenotypic traits are output by the fully convolutional neural networks. These outputs may be made directly available to a user via a GUI or may be used as inputs in further fully convolutional neural network, or other data prediction model, to generate or predict health outcomes for the animals identified and processed in the images. These predicted health outcomes may further comprise recommendations for actions to be taken based on the health outcomes, or may provide end users with the necessary information to determine health intervention actions or other actions to be taken with respect to the individual animals based on the predicted health outcomes. What the smart-barn incorporating the systems, such as systems 10, 100, and 700, provides is a non-subjective and automated solution for evaluating the visually observable phenotypes of animals and for predicting other phenotypic characteristics or traits based on the observed and processed data. This provides for more accurate, intelligent, and timely decisions regarding herd, animal, and breeding management by farmers, veterinarians, breeding managers, operations managers, and other staff in a commercial farming or breeding operation.

### EXAMPLES

### Example 1: Weight Prediction

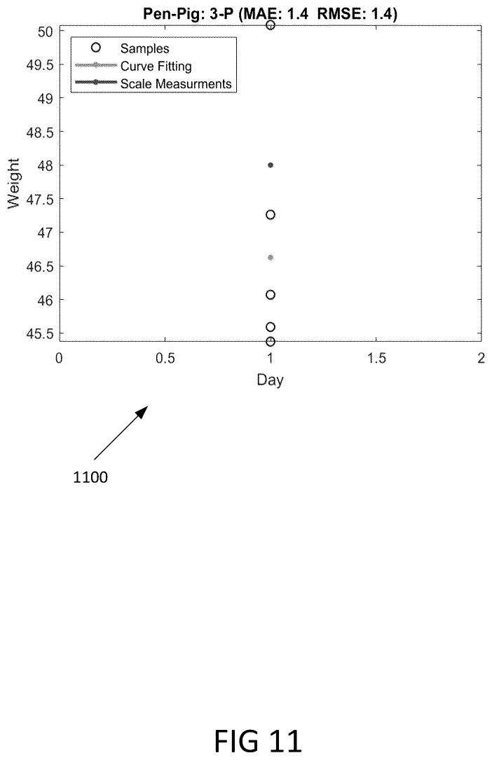

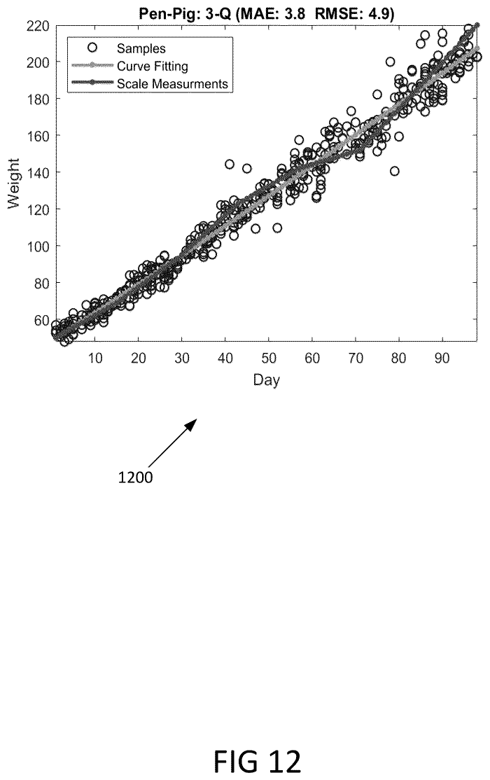

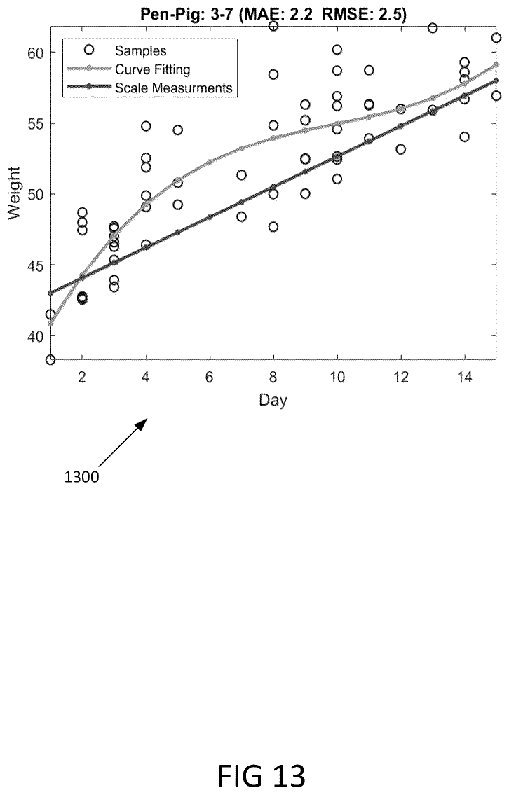

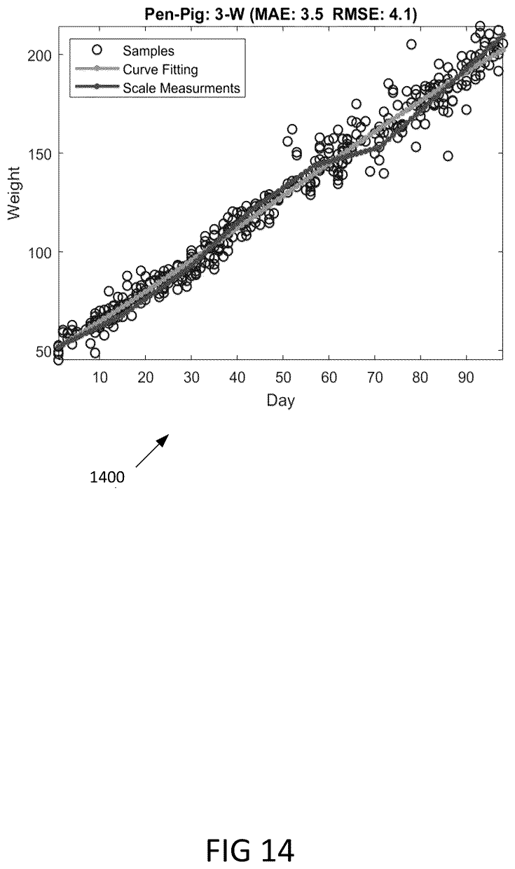

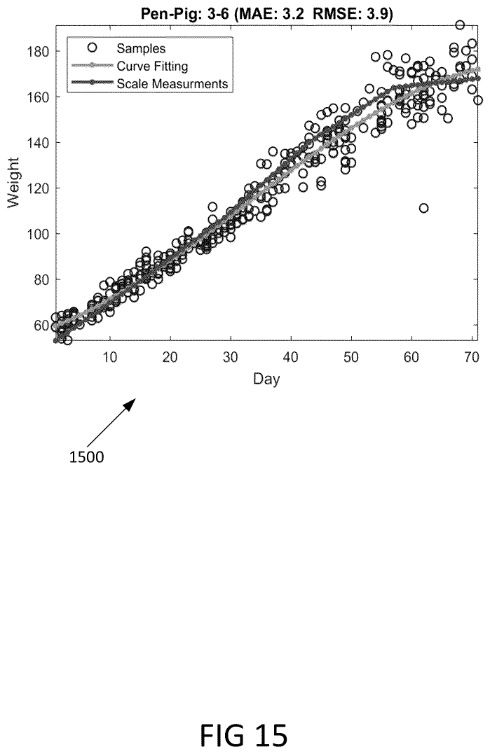

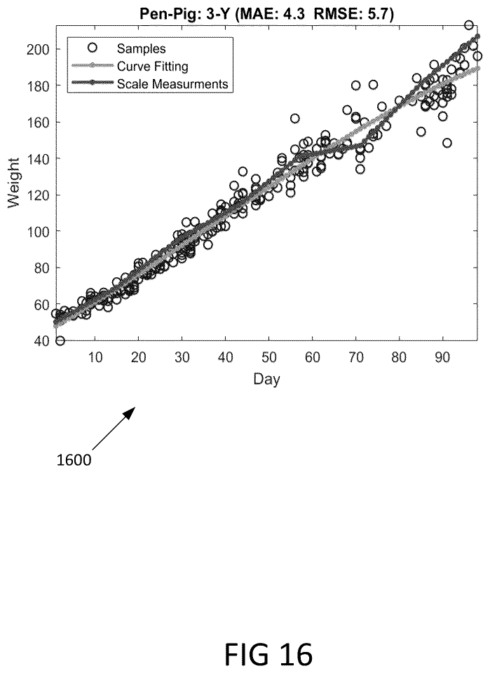

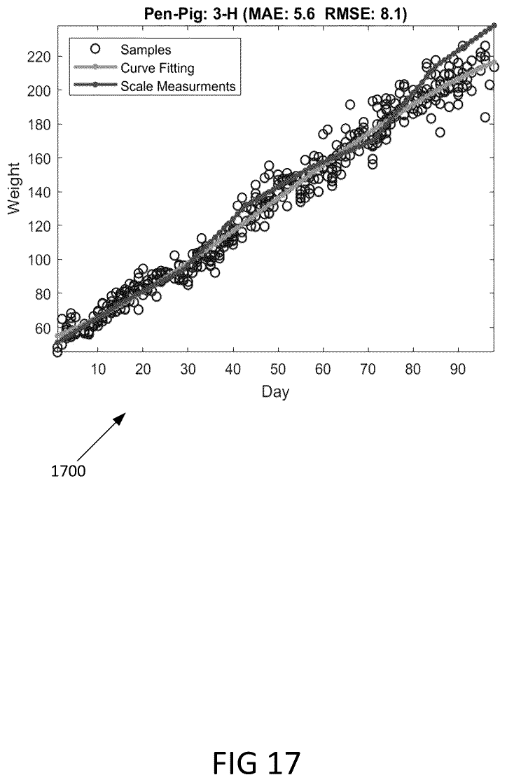

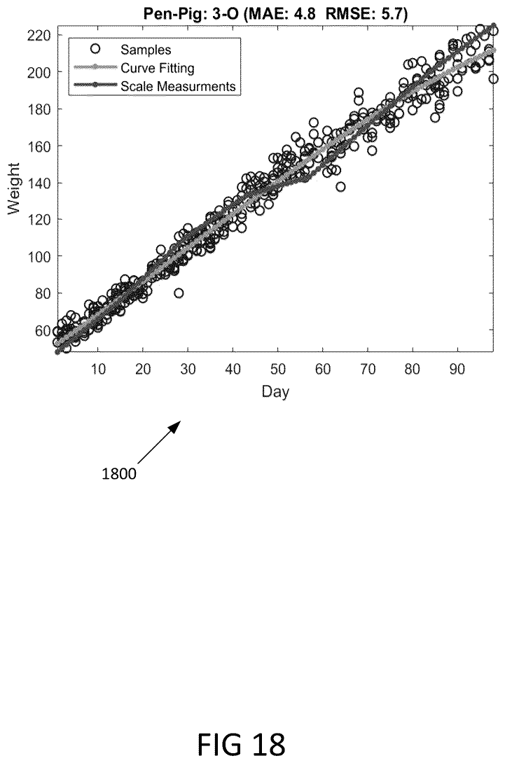

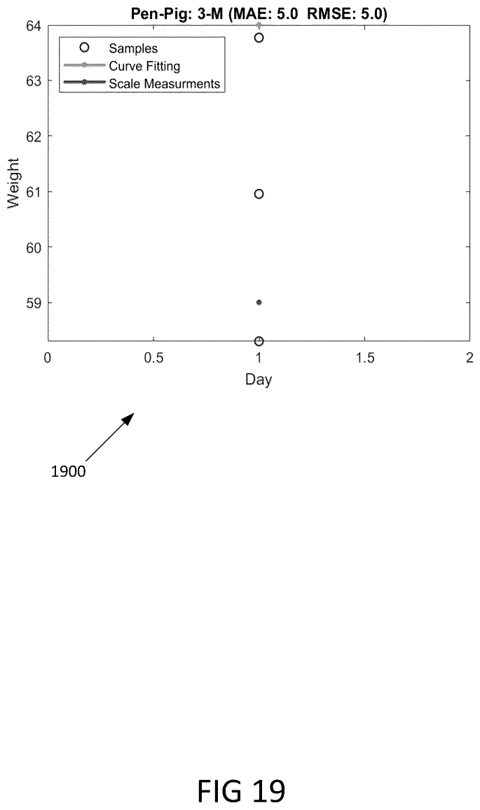

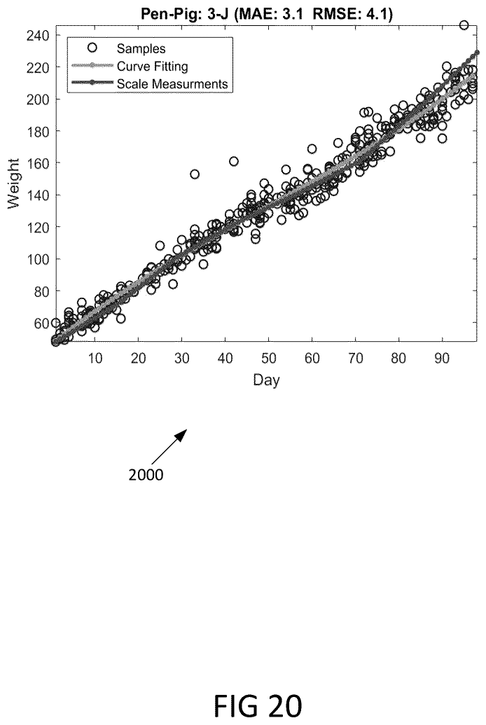

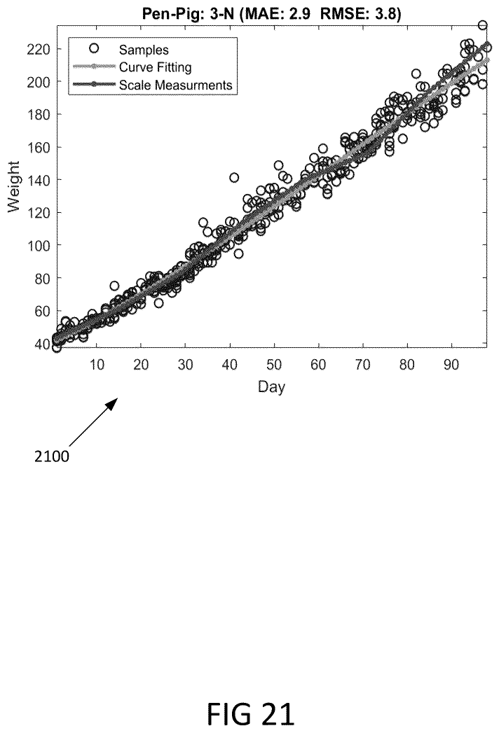

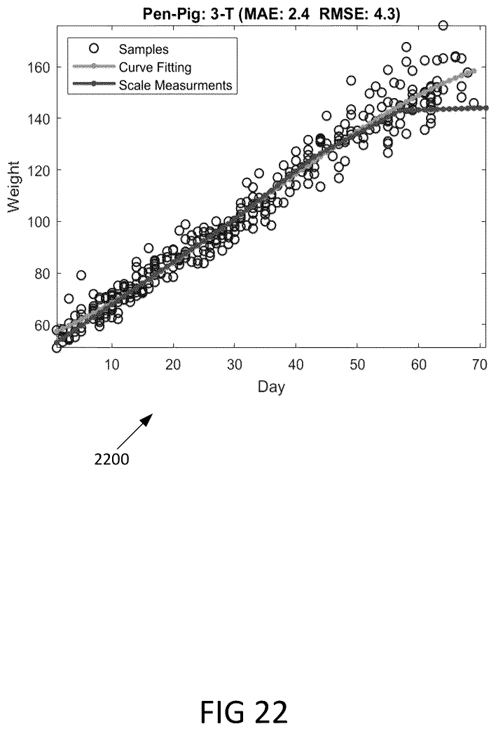

With respect to FIGS. 11-22, graphical representations of predicted weights and measured weights for a set of animals over defined time periods according to one embodiment are provided. For this example, images were extracted and cropped at tracked individual animals (as shown, for example, in FIG. 9). Corresponding time stamps were tracked to interpolated weights in a training set of 3 pens and 36 pigs used to train a fully convolutional neural network. Weight prediction was performed automatically by the fully convolutional neural network on a test pen of 12 pigs over a time period of 90 days. The predicted measurements, shown as dots, are plotted in in FIGS. 11-22 where a curve fit for the predicted sample weight measurements is provided and compared to a set of actual scale measurements over the same time frame.

### Example 2: Gait Pattern

In this example, three iterations of a video capture setup will be used to capture video for at least 400 gilts. A gait feature determination will be performed to identify what parts of the tracked gait pattern are of highest importance to be used to predict foot and leg scores. Gait feature extraction, as shown, for example in FIGS. 3 and 4, will be used on a second set of animals, such as L800 boars or gilts, to predict foot and leg scores. Boar and sow longevity studies will be used to identify gait patterns, gait scores, and foot and leg scores that serve as predictors of boar and sow longevity.

### Example 3: Gait Pattern as a Phenotypic Indication of Longevity

In this example, visual feet and leg scores were applied to gilts arriving at a sow farm before breeding of the gilts. The gilts were then evaluated to determine how long the gilts remained in production in the sow herd. Gilts having a front leg score of 7, 6, 5, and 4 had a greater productive longevity than did gilts having a front leg score of 8. For example, gilts who received a visual front leg score of 7 had a survival distribution of 0.85 at 200 days, 0.8 at 300 days, and 0.77 at 400 days compared to those with a front leg score of 8 which had a survival distribution of 0.78 at 200 days, 0.71 at 300 days, and less than 0.64 at 400 days. Gilts with a front leg score of 6, 5, and 4 each had a lower survival distribution at each of 200, 300, and 400 days compared to gilts with a front leg score of 7, but all had a higher survival distribution score at each time point compared to gilts with a front leg score of 8.

Similarly, gilts having a rear leg score of 5 or 6 had a greater productive longevity than did gilts having a rear leg score of 4 or 7. For example, gilts who received a visual rear leg score of 5 had a survival distribution of 0.84 at 200 days, 0.77 at 300 days, and 0.74 at 400 days compared to those with a rear leg score of 4 which had a survival distribution of 0.70 at 200 days, 0.66 at 300 days, and less than 0.58 at 400 days. Gilts with a rear leg score of 6 had a lower survival distribution at each of 200, 300, and 400 days compared to gilts with a rear leg score of 5, but had a higher survival distribution score at each time point compared to gilts with a rear leg score of 4 or 7.

This manual scoring showed a strong statistical correlation across multiple gilt lines between the front and rear leg scores and longevity or survival distribution. The automated, visual capture system implementing machine vision described herein was used to determine a front and rear leg score for an additional set of gilts, and the scores predicted by the system aligned with a high degree of accuracy to visual scores manually assigned to the same animals. Therefore, the machine vision system may be implemented to automatically assign a front and rear leg score to an animal which may then be used to predict a longevity for the animal and which may be used in a keep, cull, or breed decision for that animal. Suggestions as to the health outcome and an action to take based on that outcome may be automatically suggested by the system for each animal based on the automatically assigned front and rear leg scores.

In various embodiments, what is provided is a method for deriving a gait pattern in an animal, the method comprising: capturing a set of image frames of the animal, wherein the animal is in motion; determining a location of the animal for each image frame in the set of image frames; identifying a set of anatomical landmarks in the set of image frames; identifying a set of footfall events in the set of image frames; approximating a stride length for the animal based on the location of the animal in each image frame of the set of image frames, the set of anatomical landmarks, and the set of footfall events; and deriving the gait pattern based in part on the stride length, the location of the animal in each image frame of the set of image frames, the set of anatomical landmarks, and the set of footfall events.

In various embodiments, the animal is a swine.

In various embodiments, the set of image frames comprise high-resolution image frames. The high-resolution image frames comprise a resolution of at least 720 p.

In various embodiments, the motion is from a left side to a right side or from the right side to the left side in an image frame form the set of image frames, and wherein the motion is in a direction perpendicular to an image sensor.

In various embodiments, the set of image frames are captured by an image sensor. The image sensor is a digital camera capable of capturing color images. The image sensor is a digital camera capable of capturing black and white images.

In various embodiments, the set of image frames comprise a video.

In various embodiments, the method comprises determining the presence or absence of the animal in an image frame from the set of image frames.

In various embodiments, the method comprises updating a current location of the animal to the location of the animal in an image frame from the set of image frames.

In various embodiments, the method comprises determining a beginning and an end of a crossing event. The crossing event comprises a continuous set of detections of the animal in a subset of the set of image frames. The beginning of the crossing event is determined based in part on identifying that the animal occupies 20% of a left or right portion of an image frame. The end of the crossing event is determined based on identifying that the animal occupies 20% of the opposite of the left or right portion of the image frame from the beginning of the crossing event.

In various embodiments, the set of anatomical landmarks comprise a snout, a shoulder, a tail, and a set of leg joints.

In various embodiments, the method comprises interpolating an additional set of anatomical landmarks using linear interpolation where at least one of the set of anatomical landmarks could not be identified.

In various embodiments, each footfall event in the set of footfall events comprises a subset of image frames wherein a foot of the animal contacts a ground surface.

In various embodiments, approximating the stride length further comprises calculating the distance between two of the set of footfall events.

In various embodiments, the stride length is normalized by a body length of the animal.

In various embodiments, the method comprises computing a delay between a footfall event associated with a front leg of the animal and a footfall event associated with a rear leg of the animal. The method further comprises deriving a stride symmetry based in part on the delay. Deriving the gait pattern is based in part on the stride symmetry.

In various embodiments, deriving the gait pattern is based in part on a head position of the animal in a walking motion.

In various embodiments, deriving the gait pattern is based in part on a set of leg angles.

In various embodiments, the method comprises predicting a phenotype associated with the animal based on the derived gait pattern. The phenotype comprises a future health event associated with at least one leg of the animal. The method further comprises selecting the animal for a future breeding event based on the phenotype. The method further comprises identifying the animal as unsuitable for breeding based on the phenotype. The method further comprises subjecting the animal to a medical treatment based on the phenotype. The health treatment is a surgery. The health treatment is removal from a general animal population. The health treatment is an antibiotic treatment regimen. The health treatment is culling the animal.

In various embodiments, the method comprises reading identification tag associated with the animal. The capturing the set of image frames is triggered by the reading of the identification tag.

In various embodiments, the identifying the set of anatomical landmarks in the set of image frames further comprises: processing each image frame in the set of image frames using a fully convolutional neural network; identifying a nose, a mid-section, a tail, and a set of joints of interest using the fully convolutional neural network; producing a set of Gaussian kernels centered at each of the nose, the mid-section, the tail, and the set of joints of interest by the fully convolutional neural network; and extracting the set of anatomical landmarks as feature point locations from the set of Gaussian kernels produced by the fully convolutional neural network using peak detection with non-max suppression.

In various embodiments, identifying the set of anatomical landmarks in the set of image frames further comprises interpolating an additional set of anatomical landmarks, the interpolating comprising: identifying a frame from the set of image frames where at least one anatomical landmark from the set of anatomical landmarks is not detected; and interpolating a position of the at least one anatomical landmark by linear interpretation between a last known location and a next known location of the at least one anatomical landmark in the set of image frames to generate a continuous set of data points for the at least one anatomical landmark for each image frame in the set of image frames.

In various embodiments, the trained classification network is trained based in part on the stride length, the location of the animal in each frame in the set of image frames, the set of anatomical landmarks, and the set of footfall events. The trained classification network is further trained based on a delay between footfall events in the set of footfall events, a set of leg angles, a body length of the animal, a head posture of the animal, and a speed of the animal in motion. The gait score represents a time the animal is expected to be in use before culling.

In various embodiments, the method comprises: transmitting the set of image frames to a network video recorder; and storing the set of images on the network video recorder.

In various embodiments, the method comprises identifying the set of anatomical landmarks in the set of image frames by an image processing server.

In various embodiments, the method comprises identifying the set of footfall events in the set of image frames by an image processing server.

In various embodiments, the method comprises approximating the stride length for the animal based on the location of the animal in each image frame of the set of image frames, the set of anatomical landmarks, and the set of footfall events by an image processing server.

In various embodiments, the method comprises deriving the gait pattern based in part on the stride length, the location of the animal in each image frame of the set of image frames, the set of anatomical landmarks, and the set of footfall events by an image processing server.

In various embodiments, what is provided is a method of predicting at least one health outcome for an animal, the method comprising: capturing a set of high-resolution image frames of the animal, wherein the animal is in motion during the capture of the set of high-resolution image frames, and wherein the set of high-resolution image frames are captured at a rate of at least sixty times per second; determining a presence of the animal in each frame from the set of high-resolution image frames; determining a location of the animal within each frame from the set of high-resolution image frames; setting a tracked animal location as the location of the animal in a first frame in the set of high-resolution image frames where the presence of the animal is determined; updating the tracked animal location for each frame in each frame from the set of high-resolution image frames to generate a sequence of tracked animal locations; identifying a beginning and an end of an event based on the sequence of tracked animal locations, the beginning of the event comprising a first frame from the set of high-resolution image frames wherein the tracked animal location for the first frame is disposed in a left or right portion of the first frame, and the end of the event comprising a second frame from the set of high-resolution image frames wherein the tracked animal location for the second frame is disposed in an opposite portion of the second frame relative to the first frame, and wherein each frame in the set of high-resolution image frames from the first frame to the second frame comprises a set of event frames; identifying a first set of anatomical landmarks of the animal for each frame in the set of event frames; interpolating a second set of anatomical landmarks for the animal for each frame in the set of event frames, wherein the second set of anatomical landmarks comprise anatomical landmarks not in the first set of anatomical landmarks; identifying a set of footfall events from the set of event frames, a footfall event comprising a subset of frames wherein a foot of the animal contacts a ground surface; approximating a stride length for the animal based on a distance between footfall events in the set of footfall events and normalizing the stride length for the animal based on a determined body length of the animal; determining a delay between a set of front leg footfalls and a set of rear leg footfalls in the set of footfall events; deriving the gait pattern based in part on the stride length, the set of footfall events, the first set of anatomical landmarks, and the second set of anatomical landmarks, the gait pattern comprising the stride length, a symmetry of stride, a speed, a head position, and a set of leg angles; and determining a future health event for the animal based on the gait pattern, wherein the future health event is associated with an identified deficiency, abnormality, or inconsistency identified in the gait pattern.

In various embodiments, what is provided is a method of estimating a phenotypic trait of an animal, the method comprising: capturing a top-down image of the animal; bounding and isolating a central portion of the image, the central portion comprising a least distorted portion of the image; identifying a center of a torso of the animal; cropping the central portion of the image at a set distance from the center of the torso of the animal to form a cropped image; segmenting the animal into at least head, shoulder, and torso segments based on the cropped image; concatenating the at least head, shoulder, and torso segments onto the cropped image of the animal to form a concatenated image; and predicting a weight of the animal based on the concatenated image.

In various embodiments, the animal is a swine.

In various embodiments, the image comprises a greyscale image.

In various embodiments, the image comprises a set of images. The set of images comprises a video.

In various embodiments, image is captured by an image sensor. The image sensor is a digital camera. The image sensor is disposed at a fixed height with a set of known calibration parameters. The known calibration parameters comprise a focal length and a field of view. The known calibration parameters comprise one or more of a saturation, a brightness, a hue, a white balance, a color balance, and an ISO level.

In various embodiments, central portion comprising the least distorted portion of the image further comprises a portion of the image that is at an angle substantially perpendicular to a surface on which the animal is disposed.

In various embodiments, identifying the center of the torso of the animal further comprises tracking an orientation and location of the animal using a fully convolutional neural network.

In various embodiments, the method comprises extracting an individual identification for the animal. The extracting the individual identification for the animal further comprises reading a set of identification information from a tag disposed on the animal. The tag is an RFID tag or a visual tag. The extracting of the set of identification information is synchronized with the capturing of the top-down image.

In various embodiments, the cropping the central portion of the image at the set distance from the center of the torso of each of the animal further comprises: marking the center of the torso of the animal with a ring pattern; and cropping the central portion of the image at the set distance to form the cropped image. The set distance is 640×640 pixels.

In various embodiments, the segmenting the animal into the at least head, torso, and shoulder segments further comprises segmenting the animal into at least left and right head segments, left and right shoulder segments, left and right ham segments, and left and right torso segments based on the center of the torso for the animal.

In various embodiments, segmenting the animal into the at least head, torso, and shoulder segments further comprises segmenting by a fully convolutional neural network. The fully convolutional neural network is trained on an annotated image data set.

In various embodiments, segmenting is based on a ring pattern overlaid on the animal based on the center of the torso of the animal. No output may be produced where the ring pattern is not identified.

In various embodiments, the concatenating comprises stacking the at least head, shoulder, and torso segments on the cropped image in a depth-wise manner to form the concatenated image. The concatenated image comprises an input into a deep regression network adapted to predict the weight of the animal based on the concatenated image. The deep regression network comprises 9 input channels. The 9 input channels comprise the cropped image as a channel and 8 body part segments each as separate channels. The method further comprises augmenting the training of the deep regression network by randomly adjusting the position, rotation, and shearing of a set of annotated training images.

In various embodiments, the method comprises predicting a phenotype associated with the animal based on the weight of the animal. The phenotype comprises a future health event associated with the animal. The method further comprises selecting the animal for a future breeding event based on the phenotype. The method further comprises identifying the animal as unsuitable for breeding based on the phenotype. The method further comprises subjecting the animal to a medical treatment based on the phenotype. The health treatment is a surgery. The health treatment is removal from a general animal population. The health treatment is an antibiotic treatment regimen. The health treatment is culling the animal.

In various embodiments, the weight of the animal represents a time the animal is expected to be in use before culling.

In various embodiments, what is provided is method of estimating a weight of an animal based on a set of image data, the method comprising: capturing a top-down, greyscale image of at least one animal by an electronic image sensor, the electronic image sensor disposed at a fixed location, a fixed height, and with a set of known calibration parameters; bounding and isolating a central portion of the image, the central portion comprising a least distorted portion of the image that is at an angle substantially perpendicular to a surface on which the at least one animal is disposed; identifying a center of a torso of each of the at least one animal using a fully convolutional neural network; cropping the central portion of the image at a set distance from the center of the torso of each of the at least one animal; segmenting each of the at least one animal into at least left and right head segments, left and right shoulder segments, and left and right torso segments based on the center of the torso for each of the at least one animal; concatenating the at least left and right head segments, left and right shoulder segments, and left and right torso segments onto the top-down image of each of the at least one animal to form a set of concatenated images; and predicting a weight for each of the at least one animal based on the set of concatenated images.

In various embodiments, what is provided is system for determining a phenotypic trait of an animal based on a set of captured image data, the system comprising: a camera mounted above an animal retaining space and disposed at a fixed height above a central location in the animal retaining space, the camera adapted to capture and transmit an image of an animal; a horizontally-mounted camera disposed at a height aligned with a shoulder height of the animal and at an angle perpendicular to a viewing window, the horizontally-mounted camera adapted to capture and transmit a set of image frames of the animal, wherein the animal is in motion; a tag reader disposed proximate to the animal retaining space, the tag reader adapted to read a tag associated with the animal and to transmit a set of identification information read from the tag; a network video recorder comprising a storage media, the network video recorder in electronic communication with the horizontally-mounted camera and adapted to: receive the image transmitted from the camera; receive the set of image frames transmitted from the horizontally-mounted camera; and store the set of image frames and the image on the storage media; an image processing server comprising a processor and a memory, the image processing server in electronic communication with the network video recorder, and the memory comprising a first set of computer-executable instructions that when executed by the processor are adapted to cause the image processing server to automatically: request and receive the set of image frames from the network video recorder; determine a location of the animal for each image frame in the set of image frames; identify a set of anatomical landmarks in the set of image frames; identify a set of footfall events in the set of image frames; approximate a stride length for the animal based on the location of the animal in each image frame of the set of image frames, the set of anatomical landmarks, and the set of footfall events; derive the gait pattern based in part on the stride length, the location of the animal in each image frame of the set of image frames, the set of anatomical landmarks, and the set of footfall events; and store the gait pattern, the stride length, the location of the animal in each image frame of the set of image frames, the set of anatomical landmarks, and the set of footfall events in a first database, wherein each of the gait pattern, the stride length, the location of the animal in each image frame of the set of image frames, the set of anatomical landmarks, and the set of footfall events are associated with the set of identification information read from the tag; the image processing server comprising a second set of computer-executable instructions that when executed by the processor are adapted to cause the image processing server to automatically: request and retrieve the image from the network video recorder; bound and isolate a central portion of the image, the central portion comprising a least distorted portion of the image; identify a center of a torso of the animal; crop the central portion of the image at a set distance from the center of the torso of the animal; segment the animal into at least head, shoulder, and torso segments; concatenate the at least head, shoulder, and torso segments onto the top-down image of the animal to form a concatenated image; predict a weight of the animal based on the concatenated image; and store the predicted weight of the animal in a second database; and wherein a predicted phenotype for the animal is derived from the predicted weight and the gait pattern.

In various embodiments, what is provided is a system for deriving a gait pattern in an animal, the system comprising: a horizontally-mounted camera disposed at a height aligned with a centerline of the animal and at an angle perpendicular to an animal viewing window, the horizontally-mounted camera adapted to capture and transmit a set of image frames of the animal, wherein the animal is in motion; a tag reader disposed proximate to a walking path, the tag reader adapted to read a tag associated with the animal and to transmit a set of identification information read from the tag; a network video recorder comprising a storage media, the network video recorder in electronic communication with the horizontally-mounted camera and adapted to: receive the set of image frames transmitted from the horizontally-mounted camera; and store the set of image frames on the storage media; an image processing server comprising a processor and a memory, the image processing server in electronic communication with the network video recorder, and the memory comprising a set of computer-executable instructions that when executed by the processor are adapted to cause the processor to automatically: request and receive the set of image frames from the network video recorder; determine a location of the animal for each image frame in the set of image frames; identify a set of anatomical landmarks in the set of image frames; identify a set of footfall events in the set of image frames; approximate a stride length for the animal based on the location of the animal in each image frame of the set of image frames, the set of anatomical landmarks, and the set of footfall events; derive the gait pattern based in part on the stride length, the location of the animal in each image frame of the set of image frames, the set of anatomical landmarks, and the set of footfall events; and store the gait pattern, the stride length, the location of the animal in each image frame of the set of image frames, the set of anatomical landmarks, and the set of footfall events in a database, wherein each of the gait pattern, the stride length, the location of the animal in each image frame of the set of image frames, the set of anatomical landmarks, and the set of footfall events are associated with the set of identification information read from the tag.

In various embodiments, what is provided is system for estimating a weight of an animal, the system comprising: a camera mounted above an animal retaining space and disposed at a fixed height above a central location in the animal retaining space, the camera adapted to capture and transmit an image of an animal of one or more animals; a network video recorder comprising a storage media, the network video recorder in electronic communication with the camera and adapted to: receive the image transmitted from the camera; and store the image on the storage media; an image processing server comprising a processor and a memory, the image processing server in electronic communication with the network video recorder, and the memory comprising a set of computer-executable instructions that when executed by the processor are adapted to cause the processor to automatically: request and retrieve the image from the network video recorder; bound and isolate a central portion of the image, the central portion comprising a least distorted portion of the image; identify a center of a torso of the animal; crop the central portion of the image at a set distance from the center of the torso of the animal; segment the animal into at least head, shoulder, and torso segments; concatenate the at least head, shoulder, and torso segments onto the top-down image of the animal to form a concatenated image; predict a weight of the animal based on the concatenated image; and store the predicted weight of the animal in a database.

In various embodiments, what is provided is an animal health monitoring system, the system comprising: a plurality of image sensors, wherein a first image sensor from the plurality of image sensors is disposed above an animal retaining space, and wherein a second image sensor from the plurality of image sensors is disposed facing a side of the animal retaining space, the side of the animal retaining space comprising a view of the animal retaining space, the plurality of image sensors adapted to capture and transmit a set of images of the animal retaining space; a network video recorder comprising a storage media, the network video recorder in electronic communication with the plurality of image sensors and adapted to: receive the set of images from the plurality of image sensors; and store the set of images on the storage media; a phenotype prediction server comprising a processor and a memory, the phenotype prediction server in electronic communication with the network video recorder, and the memory comprising a set of computer-executable instructions that when executed by the processor are adapted to cause the processor to automatically: request and retrieve the set of images from the network video recorder; process the set of images using a fully convolutional neural network to identify a center point of the animal; identify a set of physical characteristics and anatomical landmarks of the animal based in part on the identified center point of the animal; predict a set of phenotypes associated with the animal based on the set of physical characteristics and anatomical landmarks; and present the set of phenotypes to a user in graphical user interface.

In various embodiments, what is provided is an automated smart barn, the smart barn comprising: an animal retaining space disposed in the smart barn for holding at least one animal, the animal retaining space comprising a supporting surface and a set of retaining walls; a walking path adjoining the animal retaining space, the walking path comprising a viewing widow providing a view of the walking path; a tag reader disposed proximate to the walking path, the tag reader adapted to read a tag associated with the animal and to transmit a set of identification information read from the tag, the set of identification information associated with the animal; a plurality of image sensors, wherein a first image sensor from the plurality of image sensors is disposed above the animal retaining space, and wherein a second image sensor from the plurality of image sensors is disposed facing the viewing window, the plurality of image sensors adapted to capture and transmit a set of images of the animal in the animal retaining space or walking path; a network video recorder comprising a storage media, the network video recorder in electronic communication with the plurality of image sensors and adapted to: receive the set of images from the plurality of image sensors; and store the set of images on the storage media; a phenotype prediction server comprising a processor and a memory, the phenotype prediction server in electronic communication with the network video recorder, and the memory comprising a set of computer-executable instructions that when executed by the processor are adapted to cause the processor to automatically: request and retrieve the set of images from the network video recorder; process the set of images using a fully convolutional neural network to identify a center point of the animal; identify a set of physical characteristics and anatomical landmarks of the animal based in part on the identified center point of the animal; predict a set of phenotypes associated with the animal based on the set of physical characteristics and anatomical landmarks; and present the set of phenotypes and the set of identification associated with the animal to a user in graphical user interface.

While the invention has been described by reference to certain preferred embodiments, it should be understood that numerous changes could be made within the spirit and scope of the inventive concept described. Also, the systems and methods herein are not to be limited in scope by the specific embodiments described herein. It is fully contemplated that other various embodiments of and modifications to the systems and methods herein, in addition to those described herein, will become apparent to those of ordinary skill in the art from the foregoing description and accompanying drawings. Thus, such other embodiments and modifications are intended to fall within the scope of the following appended claims. Further, although the systems and methods herein have been described herein in the context of particular embodiments and implementations and applications and in particular environments, those of ordinary skill in the art will appreciate that their usefulness are not limited thereto and that the present invention can be beneficially applied in any number of ways and environments for any number of purposes. Accordingly, the claims set forth below should be construed in view of the full breadth and spirit of the systems and methods as disclosed herein.

What is claimed is:

1. A method for deriving a gait pattern in an animal, the method comprising:
capturing a set of image frames of the animal, wherein the animal is in motion;
determining a location of the animal for each image frame in the set of image frames;
identifying a set of anatomical landmarks in the set of image frames;
identifying a set of footfall events in the set of image frames;
approximating a stride length for the animal based on the location of the animal in each image frame of the set of image frames, the set of anatomical landmarks, and the set of footfall events; and
deriving the gait pattern based in part on the stride length, the location of the animal in each image frame of the set of image frames, the set of anatomical landmarks, and the set of footfall events.

2. The method of claim 1, wherein the animal is a swine.

3. The method of claim 1, wherein the motion is from a left side to a right side or from the right side to the left side in an image frame form the set of image frames, and wherein the motion is in a direction perpendicular to an image sensor.

4. The method of claim 1, further comprising determining the presence or absence of the animal in an image frame from the set of image frames.

5. The method of claim 1, further comprising updating a current location of the animal to the location of the animal in an image frame from the set of image frames.

6. The method of claim 1, further comprising determining a beginning and an end of a crossing event comprising a continuous set of detections of the animal in a subset of the set of image frames; and
wherein the beginning of the crossing event is determined based in part on identifying that the animal occupies 20% of a left or right portion of an image frame, and wherein the end of the crossing event is determined based on identifying that the animal occupies 20% of the opposite of the left or right portion of the image frame from the beginning of the crossing event.

7. (canceled)

8. The method of claim 1, wherein the set of anatomical landmarks comprise a snout, a shoulder, a tail, and a set of leg joints.

9. The method of claim 1, further comprising interpolating an additional set of anatomical landmarks using linear interpolation where at least one of the set of anatomical landmarks could not be identified.

10. The method of claim 1, wherein each footfall event in the set of footfall events comprises a subset of image frames wherein a foot of the animal contacts a ground surface.

11. The method of claim 1, wherein approximating the stride length further comprises calculating the distance between two of the set of footfall events, and wherein the stride length is normalized by a body length of the animal.

12. The method of claim 1, further comprising computing a delay between a footfall event associated with a front leg of the animal and a footfall event associated with a rear leg of the animal; and
deriving a stride symmetry based in part on the delay, and wherein deriving the gait pattern is based in part on the stride symmetry.

13. (canceled)

14. The method of claim 1, wherein deriving the gait pattern is based in part on a head position of the animal in a walking motion or on a set of leg angles.

15. The method of claim 1, further comprising predicting a phenotype associated with the animal based on the derived gait pattern;
selecting the animal for a future breeding event based on the phenotype, identifying the animal as unsuitable for breeding based on the phenotype, or subjecting the animal to a medical treatment based on the phenotype; and
wherein the health treatment is removal from a general animal population or culling the animal.

16. (canceled)

17. (canceled)

18. The method of claim 1, further comprising reading an identification tag associated with the animal, and wherein the capturing the set of image frames is triggered by the reading of the identification tag.

19. The method of claim 1, wherein the identifying the set of anatomical landmarks in the set of image frames further comprises:
processing each image frame in the set of image frames using a fully convolutional neural network;
identifying a nose, a mid-section, a tail, and a set of joints of interest using the fully convolutional neural network;
producing a set of Gaussian kernels centered at each of the nose, the mid-section, the tail, and the set of joints of interest by the fully convolutional neural network; and
extracting the set of anatomical landmarks as feature point locations from the set of Gaussian kernels produced by the fully convolutional neural network using peak detection with non-max suppression.

20. The method of claim 1, wherein the identifying the set of anatomical landmarks in the set of image frames further comprises interpolating an additional set of anatomical landmarks, the interpolating comprising:
identifying a frame from the set of image frames where at least one anatomical landmark from the set of anatomical landmarks is not detected; and
interpolating a position of the at least one anatomical landmark by linear interpretation between a last known location and a next known location of the at least one anatomical landmark in the set of image frames to generate a continuous set of data points for the at least one anatomical landmark for each image frame in the set of image frames.

21. The method of claim 1, further comprising deriving a gait score by a trained classification network, wherein the trained classification network is trained based in part on the stride length, the location of the animal in each frame in the set of image frames, the set of anatomical landmarks, and the set of footfall events; and
wherein the trained classification network is further trained based on a delay between footfall events in the set of footfall events, a set of leg angles, a body length of the animal, a head posture of the animal, and a speed of the animal in motion.

22. (canceled)

23. The method of claim 1, further comprising:
transmitting the set of image frames to a network video recorder;
storing the set of images on the network video recorder;
identifying the set of anatomical landmarks in the set of image frames by an image processing server; and
identifying the set of footfall events in the set of image frames by the image processing server.

24. The method of claim 1, further comprising:
approximating the stride length for the animal based on the location of the animal in each image frame of the set of image frames, the set of anatomical landmarks, and the set of footfall events by an image processing server; and
deriving the gait pattern based in part on the stride length, the location of the animal in each image frame of the set of image frames, the set of anatomical landmarks, and the set of footfall events by an image processing server.

25-46 (canceled)

47. A system for determining a phenotypic trait of an animal based on a set of captured image data, the system comprising:
a camera mounted above an animal retaining space and disposed at a fixed height above a central location in the animal retaining space, the camera adapted to capture and transmit an image of an animal;
a horizontally-mounted camera disposed at a height aligned with a shoulder height of the animal and at an angle perpendicular to a viewing window, the horizontally-mounted camera adapted to capture and transmit a set of image frames of the animal, wherein the animal is in motion;
a tag reader disposed proximate to the animal retaining space, the tag reader adapted to read a tag associated with the animal and to transmit a set of identification information read from the tag;
a network video recorder comprising a storage media, the network video recorder in electronic communication with the horizontally-mounted camera and adapted to:
receive the image transmitted from the camera;
receive the set of image frames transmitted from the horizontally-mounted camera; and
store the set of image frames and the image on the storage media;
an image processing server comprising a processor and a memory, the image processing server in electronic communication with the network video recorder, and the memory comprising a first set of computer-executable instructions that when executed by the processor are adapted to cause the image processing server to automatically:
request and receive the set of image frames from the network video recorder;
determine a location of the animal for each image frame in the set of image frames;
identify a set of anatomical landmarks in the set of image frames;
identify a set of footfall events in the set of image frames;
approximate a stride length for the animal based on the location of the animal in each image frame of the set of image frames, the set of anatomical landmarks, and the set of footfall events;
derive the gait pattern based in part on the stride length, the location of the animal in each image frame of the set of image frames, the set of anatomical landmarks, and the set of footfall events; and
store the gait pattern, the stride length, the location of the animal in each image frame of the set of image frames, the set of anatomical landmarks, and the set of footfall events in a first database, wherein each of the gait pattern, the stride length, the location of the animal in each image frame of the set of image frames, the set of anatomical landmarks, and the set of footfall events are associated with the set of identification information read from the tag;
the image processing server comprising a second set of computer-executable instructions that when executed by the processor are adapted to cause the image processing server to automatically:
request and retrieve the image from the network video recorder;
bound and isolate a central portion of the image, the central portion comprising a least distorted portion of the image;
identify a center of a torso of the animal;
crop the central portion of the image at a set distance from the center of the torso of the animal;
segment the animal into at least head, shoulder, and torso segments;
concatenate the at least head, shoulder, and torso segments onto the top-down image of the animal to form a concatenated image;
predict a weight of the animal based on the concatenated image; and
store the predicted weight of the animal in a second database; and
wherein a predicted phenotype for the animal is derived from the predicted weight and the gait pattern.
